(19) United States
(12) Patent Application Publication
(10) Pub. No.: US 2023/0277696 A1
(43) Pub. Date: Sep. 7, 2023

(54) ULTRASOUND-SENSITIVE BIODEGRADEABLE MULTI-CAVITY MICRO-PARTICLES

(71) Applicant: OXFORD UNIVERSITY INNOVATION LIMITED, Oxford (GB)

(72) Inventors: James KWAN, Oxford (Oxfordshire) (GB); Umesh Sai JONNALAGADDA, Singapore (SG); Xiaoqian SU, Singapore (SG); Ipshita GUPTA, Singapore (SG)

(21) Appl. No.: 18/010,216

(22) PCT Filed: Jun. 16, 2021
(86) PCT No.: PCT/GB2021/051504

§ 371 Date: Dec. 14, 2022

(30) Foreign Application Priority Data
Jun. 16, 2020 (SG) 10202005705P
Mar. 23, 2021 (SG) 10202102971R

(51) Int. Cl. A61K 49/22

(52) U.S. Cl. CPC A61K 49/225

(57) ABSTRACT

Abstract: The present invention provides a core-shell micro-particle comprising a biodegradable polymer with at least two or more surface cavities. The present invention also provides use of the core-shell micro-particle in drug delivery, contrast enhancement, subharmonic imaging enhancement, theranostics, and/or any combination of the aforementioned applications.

### FIELD OF THE INVENTION

The invention relates to a biodegradable drug-loadable theranostic agent that enhances contrast, sustains cavitation across minutes, remains at the diseased site after exposure to ultrasound, and locally releases a payload across several days. Multi-cavity PLGA micro-particles (mcPLGA MPs) were prepared by a water/organic/water double emulsion solvent evaporation process.

### BACKGROUND

Ultrasound has garnered a lot of interest in both the diagnostic and therapeutic domains.(1-7) Not only does it allow real time imaging without exposing the patient to radiation, it is also has several therapeutic uses in tumor ablation,(3) histotripsy,(8) and better drug delivery,(9) owing to its safety, and cost and time effectiveness.

### Ultrasound for Imaging Using Contrast Agents

Conventionally, ultrasound contrast agents (UCAs) are gas filled microbubbles that act as echo-enhancers. They are used throughout the world in both clinical and research settings. (4, 6, 7, 10-16) These UCAs achieve resonance i.e. start acting as harmonic oscillators with minimum damping and have the ability to reseal post fragmentation. They are designed to act as echo-enhancers. The gas within these microbubbles has high compressibility and thus, a much higher echogenicity than the surrounding tissues. Hence, the microbubbles enhance the backscattered ultrasound signal (about 10-30 dB enhancement). This aids in contrast enhanced imaging of the blood vessels carrying these UCAs and in differentiating the vessels from the surrounding tissues. (17)

### Cavitation

As the acoustic wave propagates through a liquid media, gas molecules may coalesce to form a bubble. This bubble can further interact with the acoustic field to oscillate in size, based the rarefaction and compression phases of the field. At low acoustic intensities, the bubble will oscillate symmetrically in a process called stable cavitation. The scattered sound from stable cavitation is often used in diagnostic imaging to create specific contrast imaging modes such as subharmonic imaging, harmonic imaging, and superharmonic imaging. Stable cavitation can also perturb the surrounding fluid to create microstreams that facilitates drug transport.

Higher acoustic intensities will generate larger energy gradients, resulting in the bubble to grow. Eventually, the bubble will not retain its size and will collapse to produce inertial cavitation. The bubble collapse caused by inertial cavitation creates local physical and chemical changes. In the context of therapeutic ultrasound, these physical effects generated by inertial cavitation allow for local streaming effects to propel nanoparticles and enhance permeation of therapeutics to the desired tissue. For diagnostic imaging, the nonlinear oscillations of the bubble occur over a wide range of frequencies from subharmonics (fo/2) to second harmonics (2fo) and ultraharmonics (3fo/2) of the insonation frequency (fo) as well as its multiples. These signals can be used to create specific contrast imaging modes, such as subharmonic imaging (SHI), harmonic imaging (HI) and superharmonic imaging, respectively. (14)

Generally it is desirable for UCAs to be monodispersed in diameter and sustain cavitation for extended periods of time. Monodispersed bubbles provide a homogenous and higher echogenicity as both the resonance frequency for imaging and trigger frequency for therapy with ultrasound are size-dependent. Therefore, monodispersity is highly beneficial for ultrasound contrast agents.

It is also desirable to develop UCAs which can provide high quality images at a lower mechanical index (MI). The MI is a measure of the power of an ultrasound beam, designed as an indication of the potential of harmful, non-thermal effects on the body, from the beam. Currently, the FDA stipulates that the MI of ultrasound scanners must not exceed 1.9, with values much below this maximum threshold being preferred.

However, commercial lipid-shelled UCAs are typically polydisperse and suffer from limited stability due to coalescence and dissolution of the gas core leading to a short half-life (<10 min). UCAs with extended half-lives are desirable as they provide an extended window for accurate ultrasound imaging, thus reducing restrictive time constraints that limit the potential to obtain high quality diagnostic images.

### Drug Delivery

While the vasculature can be a minimum of 8 µm in diameter, the interstitial spacing between endothelial cells less than 100 nm in healthy tissue and sub-micron for diseased tissue. As a result, therapeutic use of UCAs is often limited due to their size and inability to permeate the endothelium.(18) In order to reduce the size of the contrast agents, one method is to manufacture perfluorocarbon microdroplets, which are capable of transitioning to gas bubbles upon ultrasound exposure and penetrate towards tumour microvasculature. It should be appreciated, however, that the acoustic intensities required for this liquid to gas transition is inversely proportional to the size of the droplets; the smaller the droplet particles, the higher the acoustic power requirement to vaporize the perfluorocarbon. To this end, solid cavitation agents have come forward as a strategy to reach smaller vasculature and inertially cavitate at lower acoustic intensities to permit extravasation across the endothelium to deep tissue.

Although efforts are being made to devise more stable UCAs, to reduce destruction by ultrasound, it is still not viable many a times. (19) Thus, to overcome these limitations, researchers have developed solid cavitation agents that are capable of stabilizing bubbles within cavities on the particles.(20, 21) These provide sustained cavitation and enable extravasation beyond the blood vessels. Solid cavitation agents typically stabilize or nucleate gas within pores or crevices on the surface or throughout the particle. There is a need for a means to not only provide targeted drug release, but to also improve the penetration and distribution of the drug to maximize therapeutic benefits. Both types of cavitation discussed above have been shown to enhance drug delivery.

It is therefore desirable to combine the targeted drug release of microbubbles with the sustained cavitation provided by solid cavitation nuclei in order to address the specific needs for addressing difficult to treat site-specific diseases. Yet, there are no degradable polymer-based drug delivery vehicles capable of nucleating cavitation from surface stabilized bubbles, despite the established research in polymer drug-delivery systems. The key challenge here is the need for surface cavities; current polymer drug-delivery vehicles are predominantly smooth spheres.

Therefore, we describe here that these needs are met with an “all-in-one” drug-loadable biodegradable solid cavitation nuclei that sustains cavitation across minutes, remain at the diseased site after exposure to ultrasound, and locally release a payload across several days. The invention also provides UCAs which exhibit a reduced cavitation threshold and increased contrast enhancement, thus displaying excellent potential as theranostic agents.

### Statements of Invention

1. A core-shell micro-particle comprising a biodegradable polymer with at least two or more surface cavities in which gas bubbles form upon mixing the core-shell micro-particle in a liquid.

2. The core-shell micro-particle according to Statement 1, wherein the gas bubbles nucleate cavitation.

3. The core-shell micro-particle according to Statement 1 or 2, wherein the biodegradable polymer is an aliphatic polyester (including but not limited to poly(lactic-co-glycolic acid) (PLGA), polylactic acid (PLA), polyglycolic acid (PGA), polycaprolactone (PCL), poly(butylene succinate) and its copolymers, poly(p-dioxanone) (PPDO), poly(hydroxybutyrate) (PHB), and polycarbonates), an aromatic co-polyester (including but not limited to poly(butylene adipate-co-terephthalate) (PBAT), polyamide and poly(ester-amide)), polyurethanes, polyanhydrides, polysaccharides (including but not limited to chitosan, cellulose, and hyaluronic acid), and blends or copolymers thereof.

4. The core-shell micro-particle according to any one of the preceding statements, wherein the surface cavities
i. are indentations on the shell; and/or
ii. form tunnels to the core; and/or
iii. result in a hierarchical porous cage-like shell around the core.

5. The core-shell micro-particle according to any one of the preceding statements, wherein the shell may further comprise one or more hydrophobic chemicals (including drugs such as sirolimus, steroids and dexamethasone).

6. The core-shell micro-particle according to any one of the preceding statements, wherein the core may further comprise one or more hydrophilic chemicals (including drugs such as peptides, proteins, and vaccines), provided that the surface cavities do not form tunnels to the core.

7. The core-shell micro-particle according to any one of the preceding statements, wherein the core-shell micro-particle is not spherical.

8. Use of the core-shell micro-particle according to any one of the preceding statements in drug delivery, contrast enhancement, subharmonic imaging enhancement, theranostics, or any combination thereof.

9. The use according to Statement 8, wherein the core-shell micro-particle is
(a) introduced into the vicinity of a biological tissue (e.g. through intravenous injection, intramuscular injection, catheter injection, or topically (through a cream, gel)); and
(b) subjected to a pressure wave such that the core-shell micro-particle is embedded into the biological tissue.

10. The use according to Statement 9, wherein the pressure wave is focused or unfocused ultrasound, or shockwaves.

11. The use according to Statement 9 or 10, wherein the biological tissue is the wall of a blood vessel, skin, or a tumor.

The invention therefore provides a core-shell micro-particle comprising a biodegradable polymer with at least two or more surface cavities. The invention is described as the following:

1. A biodegradable micro- or submicron sized particle, with at least two or more surface cavities present.

2. The surface cavities are capable of trapping gas and reducing the threshold to nucleate cavitation.

3. The resulting particle may be capable of being embedded into tissue upon exposure to ultrasound at frequencies above 100 kHz.

4. These particles may act as contrast enhancement agents for diagnostic frequencies.

5. The said particles can provide contrast to tissue ratio (CTR) values comparable to commercially available ultrasound contrast agents.

The microparticle of the invention provides an “all-in-one” drug-loadable biodegradable solid cavitation nuclei that sustains cavitation across minutes, remain at the diseased site after exposure to ultrasound, and locally release a payload across several days. Advantages of the invention are:
Multiple surface cavities to reduce the threshold to nucleate cavitation
Size and morphology is tunable.
Solid cavitation nuclei capable of being embedded into the diseased tissues upon exposure to ultrasound
Construction of a biodegradable polymer that allows its payload to be delivered at the site of the disease and not within the blood vessel network
Sustained cavitation for extended durations compared to that of microbubbles
Drug loading of hydrophobic drugs into the particle shell, hydrophilic drugs into the particle core, or a mixture of the two
Capable of providing contrast enhancement comparable to existing commercial ultrasound contrast agents
Dual modal particles for intravenous contrast enhancement and drug delivery
Potential to act as a complete theragnostic drug delivery and imaging particle

The present invention also provides a core-shell microparticle of the invention for use in drug delivery, contrast enhancement, subharmonic imaging enhancement, theranostics, and/or any combination of the aforementioned applications.

Surprisingly, it has been found that the particles described herein exhibit stable cavitation. They are therefore useful as ultrasound contrast agents. The microparticles of the invention have a low cavitation threshold, exhibit stable cavitation for extended time periods, and can be imaged using ultrasound, for example via subharmonic imaging, at low MI. A low cavitation threshold and extended stable cavitation reduce time constraints on ultrasound imaging techniques which would otherwise have to be performed quickly under time pressure before inertial cavitation occurs. This can reduce the chance that imaging will have to be repeated, or that only poor quality images will be obtainable. Combining these advantages with the ability to be imaged at a low MI, means that the microparticles of the invention can provide high quality images with reduced risk to patients.

In particular, the present invention provides microparticles which have an average particle size of from 5 to 10 µm. These microparticles provide particularly good contrast enhancement and sub-harmonic imaging enhancement, even at low MI.

### BRIEF DESCRIPTION OF THE FIGURES

FIG. 1 shows SEM images of PLGA microparticles under different concentrations of PBS and PVA. The scale bar in the top left square represents 1 µm.

FIG. 2 shows SEM images of PLGA microparticles prepared using the double emulsion-diffusion-evaporation method exhibiting a multitude of diameters and porosity proportional to the concentration of porosigen (PBS) and stabilizer (PVA), that have been labelled into 4 categories, based on their morphology and diameter namely a) non-porous hollow spheres, b) porous hollow spheres, c) large multi-cavity particles (> 5 µm in diameter), and d) small multi-cavity particles (<5 µm in diameter).

FIG. 3 shows a schematic of a HIFU setup as used in Example 2.

FIG. 4 shows pictures of (A) the agarose flow chamber and (B) the porcine artery sample chamber as used in Example 2.

FIG. 5 shows the harmonic (upper line) and broadband (lower line) cavitation intensity of PLGA microparticles exposed to 1.1 MHz HIFU.

FIG. 6 compares the effect of concentrations of PBS and PVA in the particle formulations on the rates of RhB release. The release of RhB for all formulations tested followed a rapid rate of release within the first 24 hours, after which there was a stagnation of release.

FIG. 7 shows SEM images mcPLGA MPs degrading across 15 days.

FIG. 8 shows penetration tests of “drug” loaded mcPLGA MPs. Once embedded into the agarose, the particles release the “drug”, as indicated by a decay in fluorescent intensity over 15 days.

FIG. 9 shows quantification of fluorescent intensity of FIG. 8 at both 37 C and 4 C.

FIG. 10 shows RhB-mcPLGA MPs penetration in porcine artery. (A) Control artery with no RhB-mcPLGA MPs flow and ultrasound exposure (B) Control artery with only RhB-mcPLGA MPs flow and no ultrasound exposure (C) Artery with RhB-mcPLGA MPs flow and ultrasound exposure shows penetration of RhB-mcPLGA MPs into the inner arterial wall. The dotted line boxes of A, B and C shows zoomed in images. (D) Confocal laser scanning microscope Z-stack imaging of test artery 50 um section showing location of RhB-mcPLGA MPs. These fluorescent images confirm that particles are embedded into the tissue and are not artifacts from sampling procedure. The dotted lines of D outline the endothelial and sub-endothelial region where most of RhB-mcPLGA MPs were located. RhB fluorescence is labelled red and DAPI is labelled blue.

FIG. 11 shows histopathological analysis by H&E staining of porcine arteries under (A) no RhB-mcPLGA MPs or ultrasound exposure, (B) RhB-mcPLGA MPs passed through the artery without ultrasound exposure, (C) no RhB-mcPLGA MPs with ultrasound exposure to the artery, and (D) RhB-mcPLGA MPs passed through the artery with ultrasound exposure. The fluorescence and H&E staining images of artery section with RhB-mcPLGA MPs passed through the artery with ultrasound exposure showed no signs of damage to the endothelium of the porcine artery indicating the safety of this technique.

FIG. 12 shows fluorescence images of porcine arteries under (Top panel) RhB-mcPLGA MPs passed through the artery without ultrasound exposure, and (Bottom panel) RhB-mcPLGA MPs passed through the artery with ultrasound exposure. The fluorescence images of artery section with RhB-mcPLGA MPs passed through the artery with ultrasound exposure show no signs of damage to the endothelium of the porcine artery, indicating the safety of this technique.

FIG. 13 shows a fluorescent image of a 3D foam cell spheroid exposed to DAPI-RhB-mcPLGA MPs and ultrasound one day after remote implantation.

FIG. 14 shows oil Red O staining of cytoplasmic lipid droplets showing the effect of Dex on lipid accumulation in foam cell spheroids.

FIG. 15 shows a schematic representation of the therapeutic ultrasound experimental set-up as used in Example 3.

FIG. 16 shows representative images of the normalized spectral density curves for three different shapes of particles, namely the a) spherical and both the b) small and c) large multicavity particles. As is seen, the multicavity particles (see b & c) transitioned from stable to inertial cavitation at much lower pressures in comparison to the nonporous spherical variants.

FIG. 17 shows the intensity of harmonic (upper line) and broadband (lower line) emissions observed for all microparticle formulations, and their dependence on both the diameter of the microparticle and acoustic intensity.

FIG. 18 shows the estimated dependence of acoustic pressure amplitude required to achieve 50% probability of (a) harmonic and (b) broadband cavitation on the diameter of particles. The dependence on particle diameter can be observed indicating the effect of Laplace pressure on cavitation threshold most strikingly for the porous hollow spheres. tested.

FIG. 19 shows the probability of cavitation for all microparticle formulations, and the dependence of probability of cavitation on both the diameter of the microparticle and acoustic intensity. Left hand lines indicate stable cavitation thresholds, right hand lines indicate inertial cavitation thresholds.

FIG. 20 shows a) a schematic representation of the diagnostic ultrasound experimental setup. b) a schematic for selection of region of interest (ROI) for vessel and tissue. CTR analysis was done by calculating the average of pixel intensity in the four tissue and two vessel ROIs selected. This was done to minimize variability.

FIG. 21 shows samples as imaged with the diagnostic ultrasound scanner and the corresponding contrast to tissue ratio (CTR) values in dB with reference to deionized water. As is seen, highest enhancement (> 20 dB) was observed for the larger multicavity particles (diameter > 5 µm) i.e., category (c): the right top corner and in the sample in the middle of the matrix.

FIG. 22 shows the measured CTR for representative microparticles from the different morphology groups (2 µm in diameter smooth spheres (labelled non-porous spheres), 2 µm in diameter multi-cavity microparticles (labelled smaller multicavity particles), and 6 µm in diameter multi-cavity microparticles (labelled larger multicavity particles)) in addition to deionized water for increasing input pressures from 10% to 100% power (corresponding MI values of 0.11 to 1.1).

FIG. 23 shows the representative images and CTR analysis for Example 5, at varying concentrations and volumes of stabiliser (PVA) and porosigen (PBS).

FIG. 24 shows the subharmonic imaging results of Example 6 wherein a) shows a regular B mode image at MI 1.4 wherein hollow spheres can be seen moving in, b) shows hollow spheres imaged at MI 0.4, c) shows 2 µm particles are observed at MI 0.4, d) shows 5.8 µm particles are observed at MI 0.4.

FIG. 25 shows the dependence of CTR to acoustic power is demonstrated for hollow spheres (lower line), 2 µm particles (middle line) and 5.8 µm particles (upper line), as determined by Example 6.

### DETAILED DESCRIPTION OF THE INVENTION

The present invention provides a core-shell microparticle comprising a biodegradable polymer with at least two or more surface cavities.

In one embodiment, a biodegradable microparticle comprises:
a) a plurality of surface cavities; and
b) a gas pocket present in some or all of the surface cavities.

### Particles of the Invention

The core-shell microparticle of the invention is a multi-cavity particle comprising a biodegradable shell surrounding a core, wherein the core is optionally a hollow core. In some embodiments the core-shell microparticle is not spherical. In one embodiment, each biodegradable microparticle may comprise between 2 to 5 surface cavities. The multicavity particles may have cavities in a variety of different forms. For instance, the cavities may be in the form of cups, pores, or tunnels which go through the particles. Further, the surface cavities of the invention may be indentations on the shell, and/or they may form a hierarchical porous shell with the hollow core, wherein the resultant hierarchical porous shell may be cage-like and/or they may form tunnels to the core.

The multi-cavity structure may contain from 2 to 20 cavities, preferably from 2 to 10 cavities, more preferably from 2 to 5 cavities.

Having multiple surface cavities reduces the threshold to nucleate cavitation. A lower threshold to nucleate cavitation is advantageous, as it allows ultrasound with a lower mechanical index to be used for imaging. As discussed above, the MI is a measure of the power of an ultrasound beam, designed as an indication of the potential for harmful, nonthermal effects on the body from the beam. Currently, the FDA stipulates that the MI of ultrasound scanners must not exceed 1.9, with values much below this maximum threshold being preferred. Therefore, the multiple surface cavities of the invention, which enable imaging with lower MI, is highly advantageous, reducing risk to the body associated with exposure to ultrasound

The core-shell microparticle of the invention typically has an average diameter of 10 µm or less. In some embodiments it has a diameter of from 5 µm to 10 µm, preferably 5 µm to 6 µm These embodiments of the invention are more suitable for diagnostic uses such as ultrasound contrast agents, including sub-harmonic contrast agents. In some embodiments the core-shell microparticle of the invention has a diameter of 5 µm or less, preferably from 2 µm to 5 µm. A microparticle as described herein may have a diameter of less than 2 µm, for example less than one micron. Typically, a microparticle has a diameter of at least 0.1 µm, preferably at least 0.5 µm. A suitable microparticle therefore has a diameter of from 0.1 to 10 µm, preferably 0.1 to 6 µm or 0.5 to 6 µm. Many methods are available for measurement of particle diameter, for example SEM or dynamic light scattering.

Preferably, the microparticles of the invention are highly monodispersed. Typically the polydispersity index (PDI) of the particles of the invention, or of a composition comprising the particles of the invention, is 0.3 or less, preferably 0.2 or less.

Typically the cavities of the multi-cavity microparticles have an average diameter of from 0.1 to 1.0 µm, for example from 0.4 µm to 0.8 µm, preferably from 0.65 µm to 0.75 µm. Diameter of cavities may be measured by, for instance, SEM.

The shell of the core-shell microparticle of the invention typically comprises a biodegradable polymer. The biodegradable polymer of the core-shell microparticle may be an aliphatic polyester, an aromatic copolyester, polyamide, poly(ester-amide), polyurethanes, polyanhydrides, polysaccharides, and blends or copolymers of the aforementioned examples.

When the biodegradable polymer is an aliphatic polyester, it is preferably poly(lacticco-glycolic acid) (PLGA), polylactic acid (PLA), polyglycolic acid (PGA), polycaprolactone (PCL), poly(butylene succinate) and its copolymers, poly(p-dioxanone) (PPDO), poly(hydroxybutyrate) (PHB), polycarbonates, or blends or copolymers thereof. When the biodegradable polymer is an aromatic copolyester, it is preferably poly(butylene adipate-co-terephtalate) (PBAT) or a copolymer thereof. Preferably, the biodegradable polymer is an aliphatic polyester, or blend or copolymer thereof. More preferably, the biodegradation polyester is poly(lactic-co-glycolic acid) (PLGA), or a blend or copolymer thereof. Preferably the PLGA polymer comprises a blend of lactic acid to glycolic acid of from 25:75 to 75:25, preferably from 40:60 to 60:40.

The biodegradable nature of the materials used for the present invention is highly advantageous as it prevents build-up of non-biodegradable polymers in the body, which can be toxic or harmful upon accumulation. That the microparticles degrade in the body over time is also advantageous as it allows for the slow release in targeted areas of drugs which may be contained in the polymer shell.

In one embodiment, each biodegradable microparticle may comprise at least one drug in the microparticle shell. In some embodiments the microparticle shell of the present invention comprises at least one hydrophobic drug. In some embodiments the drug is a drug for use in the treatment of cancer, atherosclerosis, thrombolysis (blood clot destruction), blood-brain barrier disruption, arteries damaged post angioplasty and restenosis of blood vessels. In some embodiments the at least one drug may be one or more of a drug for use in treatments of atherosclerosis or restenosis of blood vessels. In some embodiments the at least one drug may be one or more of a chemotherapy agent, such as a taxane; an immunosuppressant, such as sirolimus; or a steroid, such as a corticosteroid, such as dexamethasone. Preferably, the at least one hydrophobic drug comprises one or more of sirolimus and a steroid, such as dexamethasone.

In some embodiments the microparticle shell does not comprise a drug. In some embodiments, the microparticle shell consists essentially of, preferably consists of, one or more biodegradeable polymers, such as the biodegradeable polymers listed herein.

In one embodiment, each biodegradable microparticle may comprise of at least one drug in the microparticle core. Preferably, the at least one drug is a hydrophilic drug. More preferably the drug is a peptide or protein based drug, or a vaccine. In some embodiments, the core of the biodegradable microparticle of the invention comprises at least one drug, provided that the surface cavities of the microparticle do not form tunnels to the core.

In certain embodiments, each biodegradable microparticle may comprise of at least one drug in the microparticle shell and at least one drug in the microparticle core

### Synthesis

Typically, the microparticles of the present invention are prepared using a double emulsion procedure. The first step of the synthesis of the microparticles of the invention typically comprises dissolving the at least one biodegradable polymer, and optionally the at least one drug as discussed above, in an appropriate solvent to produce an organic phase. In an exemplary embodiment this first step comprises dissolving PLGA in dichloromethane (DCM).

Typically this step is followed by addition of an aqueous phase to the organic phase and mixing (for example by sonication) to form a water-in-oil (W/O) emulsion. The aqueous phase may comprise a porosigen, for example phosphate buffered saline (PBS).

The next step to obtaining the microparticles of the invention is typically to combine the W/O emulsion with a further aqueous phase and homogenise the resulting mixture to produce a W/O/W emulsion. The further aqueous phase typically comprises a stabiliser such as poly(vinyl alcohol) (PVA). The organic solvent may then be allowed to evaporate from the W/O/W emulsion.

In order to trap gas, the particles of the invention are typically dried in air (or an alternative gas) to form a gas bubble. The particles may then be re-suspended. The drying and re-suspending process allows the particles of the invention to cavitate.

The microparticles of the invention can then be collected, for example by centrifugation, and optionally washed, for example with distilled water, and redispersed.

The microparticles of the invention may then be lyophilised to achieve a dried powder for long term storage.

### Tuneability

The core-shell microparticle of the invention has the advantage of tuneable size and morphology. This has allowed the present inventors to hone the parameters of the microparticles to optimise factors such as the cavitation threshold. This also allows for more flexible uses of the technology, which can be adapted for smaller particles (particle diameter < 5 µm) more suitable for therapeutic purposes, and larger particles (particle diameter > 5 µm) which are more suitable for diagnostics. Smaller variants would be ideal for use in the therapeutic domain given their favourable size, and the larger particles would nucleate bubbles with a larger scattering cross section and behave as better contrast agents but achieve poorer perfusion.

The shape and dimensions of the microparticle of the invention may be controlled by varying the concentration and composition of the various reagents used in synthesis of the particles.

The first aqueous phase typically comprises a porosigen. A porosigen may be any suitable material which will disperse or degrade to leave a porous network. For example, the porosigen may be a salt solution, such as a solution of a sodium and/or potassium salt, e.g. sodium chloride, potassium chloride, sodium phosphates such as sodium dihydrogen phosphate, potassium phosphates such as potassium dihydrogen phosphate, or mixtures thereof. PBS solution, which is a mixture of such salts, is a preferred porosigen. Suitable concentrations of porosigen in the first aqueous phase are from 0.01 to 0.5 M, e.g. 0.01 M to 0.2 M.

The shape of the microparticles of the invention can be controlled by adjusting the composition of the first aqueous phase, such as by adjusting the concentration of the porosigen. Generally the inventors have found that increasing the porosigen (typically the salt) concentration of the first aqueous phase produces microparticles of the invention with greater numbers and/or sizes of cavities. For example, using a first aqueous phase which is PBS having a 0.01 M salt concentration may produce microparticles with small, infrequent surface pores that do not penetrate the full depth of the shell, while using a first aqueous phase which is PBS have 0.1 M salt concentration may result in particles with higher numbers of cavities, as well as deeper cavities, possibly including pores and/or tunnels as well as surface cavities.

The size of the microparticles of the invention can also be controlled by adjusting the salt concentration of the porosigen of the aqueous component. Typically, the present inventors have found that an increased concentration of salt, such as PBS, in the porosigen results in a microparticles forming with a larger diameter. For example, using a first aqueous phase which is PBS having a 0.01 M salt concentration, and using 1 wt % PVA in the further aqueous phase, PLGA microparticles generally formed with an average diameter of roughly 2 µm. However, in an alternative exemplary synthesis it was found that with a first aqueous phase which is PBS have 0.1 M salt concentration, and 1 wt % PVA in the further aqueous phase, the PLGA microparticles generally formed with an average diameter of roughly 6 µm.

The further aqueous phase may comprise a stabilising agent. The stabilising agent may be any suitable material which can stabilise the water-oil interface. PVA is a preferred stabilising agent. A suitable amount of stabilising agent is from 1 to 10 wt% in the further aqueous phase.

The shape of the microparticle can be controlled by adjusting the concentration of the stabilising agent, such as by adjusting the weight percentage of PVA used. Conversely to the concentration of the porosigen, the present inventors found that increasing the weight percentage of stabilising agent used in the synthesis of the microparticles of the invention, reduced the depth of the surface cavities that formed.

The size of the microparticle of the invention can also be controlled by adjusting the concentration of the stabilising agent, such as by adjusting the weight percentage of PVA in the further aqueous solution. The present inventors have found that a higher weight percentage of stabiliser reduces the average diameter of microparticles synthesis. For example, in an exemplary embodiment of the invention, using a first aqueous phase which is PBS having a 0.2 M salt concentration, and using 1 wt % PVA in the further aqueous phase, PLGA microparticles generally formed with an average diameter of roughly 6 µm. In another exemplary embodiment of the invention, wherein the conditions and reagents were the same except that 10 wt% PVA was used, the PLGA microparticles were found to form with an average diameter of roughly 2 µm.

An ability to finely tune the size and morphology of the particles in the above ways is extremely advantageous, as it allows for microparticles of the invention with preferential properties for use in drug delivery, theranostics, or imaging to be synthesised.

### Incorporation of Drugs

In some embodiments of the invention one or more drugs can be incorporated into either the core or the shell of the microparticle. In some embodiments this incorporation is achieved by dissolving the one or more drugs in the organic phase and/or an aqueous phase. Preferably, the one or more drugs is dissolved in the organic phase. Typically hydrophobic drug(s) are incorporated. Once the drug has been dissolved in the organic phase, following the steps described above, the one or more drugs will be located in the biodegradable polymer shell of the fully formed core-shell microparticle. For example, the one or more drugs may be entrapped within the polymer network of the PLGA polymer.

The present inventors have found that the in embodiments which contain one or more drugs, the microparticles of the present invention can be adapted to control the release rates of the one or more drugs. For example the inventors found that the release rate could be increased by increasing percentage weight of stabilising component (such as PVA) in the reaction mixture. Such tuneability is extremely desirable as it allows more versatile usage of the microparticles of the invention, such that they can be used to adjust dosage to fit the desired use.

### Uses

In some embodiments the invention provides the core-shell microparticles for use in drug delivery, contrast enhancement, subharmonic imaging enhancement, theranostics, and/or any combination of the aforementioned applications. In some embodiments the invention provides use of the core-shell micro-particle in drug delivery, contrast enhancement, subharmonic imaging enhancement, theranostics, and/or any combination of the aforementioned applications.

In particular, the present invention provides contrast enhancement comparable to existing ultrasound contrast agents, as well as providing the potential for dual modal particles for both intravenous contrast enhancement and drug delivery. This means the present invention has the potential to provide a complete theranostic drug delivery and imaging particle.

The present invention also provides a method of delivering drugs, contrast enhancement, subharmonic imaging enhancement, theranostics and/or any combination of the aforementioned applications, which method comprises administering to a subject an effective amount of a microparticle according to the invention. In some embodiments, the method is a method of drug delivery comprising
(a) administering an effective amount of the core-shell micro-particle according to any one of the claims 1 to 7, which core shell microparticle contains one or more drugs; and
(b) subjecting the core-shell microparticle or ultrasound contrast agent to a pressure wave at the site for drug delivery, such that the core-shell microparticle or ultrasound contrast agent is embedded into biological tissue at the site.

The administration may be to a blood vessel or to the skin. The method may further comprise subjecting the site to ultrasound imaging, as described herein.

Also provided is the use of the microparticles of the invention in the manufacture of a composition for drug delivery, contrast enhancement, subharmonic imaging enhancement, theranostics and/or any combination of the aforementioned applications.

In some embodiments the core-shell microparticle of the invention is for use as described above wherein the core-shell micro-particle is
(a) introduced into a blood vessel (e.g., through intravenous injection, local deliver, intramuscular injection, catheter injection, etc.) or topically (e.g., through a cream, gel, etc.); and
(b) subjected to a pressure wave (e.g., ultrasound, focused ultrasound, shockwaves, etc.) such that the core-shell micro-particle is embedded into biological tissue (e.g., the wall of the blood vessel, skin, tumor, etc.).

This has the advantage of providing the microparticles of the invention at a desired target site, for instance for delivering drugs to a target site, or for contrast enhancement at the target site.

Typically, the microparticles of the invention are delivered via injection, for example by intravenous injection or via a catheter, typically directly into a blood vessel. However, alternative means of administration such as subdermal injection or intramulscular injection are also possible. In the case of injection, the particles are typically administered in the form of an aqueous solution or suspension. In some embodiments the microparticles for use in any of the uses described above may be administered non-invasively, such as in a cream or gel.

Following administration, if it is desired to deliver the microparticles to a selected site, for example a target site for delivery of drug or for contrast imaging, the desired site is subject to a pressure wave. The pressure wave as described above is typically focused or unfocused ultrasound or shockwaves, typically it is high intensity focused ultrasound. Conventional high intensity focused ultrasound may be used.

The solid cavitation nuclei of the present invention are capable of being embedded into tissue (e.g. diseased tissues) upon exposure to ultrasound. For example, under inertial cavitation, bubble (gas pocket) collapse may create local physical and chemical changes. In the context of therapeutic ultrasound, these physical effects generated by inertial cavitation allow for local streaming effects to propel nanoparticles and enhance permeation of therapeutics to the desired tissue. Stable cavitation can also aid in embedding of particles through enhanced microstreaming or micromixing, drug delivery to tissue and cells, and reversible permeation of membranes such as the blood-brain barrier. Such factors allow the payload of the biodegradable polymer as used in the invention to be delivered at the site of the disease and not within the blood vessel network.

Thus, on application of ultrasound, the microparticles of the invention may undergo stable and/or inertial cavitation. The particles are thus capable of being embedded into the wall of a blood vessel, skin, or tumour, for example particles are capable of being embedded in the sub-endothelial region.

Currently, cavitation nuclei being explored for their ability to deliver a therapeutic are limited to either gaseous microparticles, phase change droplets, or non-degradable solid nuclei. Our technology provides an improvement on all of these particles. With regard to microbubbles and phase-change droplets, our particles are not destroyed during ultrasound exposure and sustain cavitation for nearly 10 minutes. Furthermore, our particles are capable of being embedded into tissue for direct delivery of the therapeutic at the site of injury. With regard to solid cavitation nuclei (such as nanocups or mesoporous silica), the proposed particles are comprised of a biodegradable material. This enables its contents to be released over long periods of time. Therefore, these particles will have an advantage in delivering therapeutic agents to nearly any disease whereby drug distribution is a challenge without having to be concerned with the side effects of a non-degradable particle being situated at the site of injury.

This invention has use in nearly any healthcare application whereby there is a need to improve the local distribution of a therapeutic. Below is a list of some selected drug delivery applications:
Cancer therapy
Atherosclerosis
Transdermal delivery
Thrombolysis (blood clot destruction)
Blood-brain barrier disruption
Anti-inflammatory drug delivery to damaged arteries post angioplasty
Prevention of restenosis through the delivery of anti-proliferation drugs (e.g., sirolimus)

Where the microparticles of the invention are used for drug delivery, the amount of drug to be delivered can be determined by the skilled person using known dosages for the drug to be incorporated.

These microparticles are also able to act as contrast enhancement agents, giving CTR values as high as 40 dB (comparable to existing commercially available ultrasound contrast agents). However, unlike the existing contrast agents (microbubbles), these particles have the additional capability of being loaded with a high concentration of therapeutic agents and achieve sustained drug release along with being biodegradable.

Thus, in particular embodiments the present invention provides core-shell microparticles of the invention for use in ultrasound contrast enhancement. The invention also provides an ultrasound contrast agent comprising a core-shell microparticle as described herein. Preferably, the ultrasound contrast agent is a subharmonic imaging contrast agent. The particles may be provided together with one or more pharmaceutically acceptable carriers or diluents.

The invention also provides a method of ultrasound contrast enhancement comprising delivering the microparticles of the invention to a site which is to be imaged and subjecting the site to ultrasound imaging. Also provided is the use of microparticles as described herein in the manufacture of a composition for use in ultrasound contrast enhancement.

If desired, the delivery of the particles to the site for imaging may be carried out as described above, i.e. wherein the core-shell micro-particle is
(a) introduced into a blood vessel (e.g., through intravenous injection, local deliver, intramuscular injection, catheter injection, etc.) or onto the skin, i.e. topically (e.g., through a cream, gel, etc.); and
(b) subjected to a pressure wave (e.g., ultrasound, focused ultrasound, shockwaves, etc.) such that the core-shell micro-particle is embedded into biological tissue (e.g., the wall of the blood vessel, skin, tumor, etc.).

Suitable delivery to a target site for imaging is typically as described herein.

In preferred embodiments the ultrasound imaging is subharmonic imaging. The ultrasound contrast enhancement or subharmonic imaging may be carried out at an MI of 1.4 or less, preferably 1.0 or less, more preferably 0.6 or less, 0.8 or less, or 0.4 or less. Image enhancement at such low MI decreases risk associated with exposure of the human body to ultrasound.

he core-shell microparticles of the invention having an average diameter of 5 µm to 10 µm, preferably 5 µm to 6 µm, have been shown to be especially effective ultrasound contrast agents, providing high CTR values. This is in part due to the extended stable cavitation exhibited by these particles. However, stable cavitation has also been observed for the present invention in particles of diameters of < 5 µm. The presence of stable cavitation from the microparticles at clinically relevant diameters suggests the potential for these microparticles to be used for imaging in various modes including harmonic and subharmonic imaging.

The core-shell microparticles of the invention are also particularly useful in subharmonic imaging, as the enhancement provided by the particles of the invention is especially well defined, even at low mechanical index. The core-shell microparticles of the invention having an average diameter of 5 µm to 10 µm, preferably 5 µm to 6 µm, have also been shown to be especially effective as subharmonic contrast agents.

The particles used for ultrasound contrast enhancement may be particles which do not carry any drug. Thus, the particles may consist of, or consist essentially of, biodegradeable polymer. In this instance, the particles may be solely for use in ultrasound contrast enhancement. Alternatively the particles may additionally comprise one or more drugs, such that the particles may deliver drug to the target site as well as acting as ultrasound contrast agents (UCAs).

These particles can be used as UCAs and given their proven sustained drug release profile, they can have a true theragnostic utility, where they can be used in treatment and simultaneously be imaged to study the location and release of drugs over time. Thus, in some embodiments the present invention provides core-shell microparticles for use in theranostic treatments. A theranostic treatment refers to a singular treatment which can both image and treat a condition.

Thus, the core-shell microparticles may be used in methods which combine one or more of the above described uses.

### BIBLIOGRAPHY

1. Lipsman N, Meng Y, Bethune AJ, Huang Y, Lam B, Masellis M, et al. Blood-brain barrier opening in Alzheimer’s disease using MR-guided focused ultrasound. Nature communications. 2018; 9(1):2336(2041-1723 (Electronic)).

1. Hg M, Am M, Husseini GA. Review on triggered liposomal drug delivery with a focus on ultrasound. Curr Cancer Drug Targets. 2015;15(4)(1873-5576 (Electronic)):282-313.

2. Orsi F, Zhang L, Arnone P, Orgera G, Bonomo G, Vigna P, et al. High-intensity focused ultrasound ablation: effective and safe therapy for solid tumors in difficult locations. AJR American journal of roentgenology. 2010;195(3):W:245-52.

3. Dave JK, Halldorsdottir VG, Eisenbrey JR, Raichlen JS, Liu J-B, McDonald ME, et al. Subharmonic microbubble emissions for noninvasively tracking right ventricular pressures. American Journal of Physiology-Heart and Circulatory Physiology. 2012;303(1):H126-H32.

4. Eisenbrey JR, Dave JK, Halldorsdottir VG, Merton DA, Miller C, Gonzalez JM, et al. Chronic Liver Disease: Noninvasive Subharmonic Aided Pressure Estimation of Hepatic Venous Pressure Gradient. Radiology. 2013;268(2):581-8.

5. Forsberg F, Piccoli CW, Merton DA, Palazzo JJ, Hall AL. Breast lesions: Imaging with contrast-enhanced subharmonic US - Initial experience. Radiology. 2007;244(3):718-26.

6. Gupta I, Forsberg F, Stanczak M, Sridharan A, Dave J K, Liu CJB, et al., editors. Improved measurement of portal pressures using subharmonic contrast imaging and pulse shaping.. Prog RSNA; 2016; Chicago.

7. Roberts WW, Hall T, Ives K, Wolf JJ, Fowlkes J, Cain CA. Pulsed cavitational ultrasound: a noninvasive technology for controlled tissue ablation (histotripsy) in the rabbit kidney. The Journal of urology. 2006;175(2)(0022-5347 (Print)):734-8.

8. Hernot S, Klibanov AL. Microbubbles in ultrasound-triggered drug and gene delivery. Advanced drug delivery reviews. 2008;60(10)(0169-409X (Print)): 1153-66.

9. Bogers HA, Sedelaar JP, Beerlage HP, de la Rosette JJ, Debruyne FM, Wijkstra H, et al. Contrast-enhanced three-dimensional power Doppler angiography of the human prostate: correlation with biopsy outcome. Urology. 1999;54(1):97-104.

10. Calliada F, Campani R, Bottinelli O, Bozzini A, Sommaruga MG. Ultrasound contrast agents: basic principles. Eur J Radiol. 1998 (May;27 Suppl 2:S157-60.).

11. Cohen S. AAK, Wheatley M., Allcock H. R., Langer R. S., inventorGas-filled polymeric microbubbles for ultrasound imaging 1996.

12. Dave JK, Halldorsdottir VG, Eisenbrey JR, Forsberg F. Processing of Subharmonic Signals from Ultrasound Contrast Agents to Determine Ambient Pressures. Ultrason Imaging. 2012;34(2):81-92.

13. Forsberg F, Liu JB, Shi WT, Furuse J, Shimizu M, Goldberg BB. In vivo pressure estimation using subharmonic contrast microbubble signals: Proof of concept. IEEE transactions on ultrasonics, ferroelectrics, and frequency control. 2005;52(4):581-3.

14. Forsberg F, Liu JB, Shi WT, Furuse J, Shimizu M, Goldberg BB. In vivo perfusion estimation using subharmonic contrast microbubble signals. J Ultrasound Med. 2006;25(1):15-21.

15. Forsberg F, Shi WT, Goldberg BB. Subharmonic imaging of contrast agents. Ultrasonics. 2000;38(1-8):93-8.

16. Stride EP, Coussios CC. Cavitation and contrast: the use of bubbles in ultrasound imaging and therapy. Proceedings of the Institution of Mechanical Engineers Part H-Journal of Engineering in Medicine. 2010;224(H2):171-91.

17. Zhao Y-Z, Du L-N, Lu C-T, Jin Y-G, Ge S-P. Potential and problems in ultrasoundresponsive drug delivery systems. International journal of nanomedicine. 2013;8:1621-33.

18. Thakur SS, Ward MS, Popat A, Flemming NB, Parat M-O, Barnett NL, et al. Stably engineered nanobubbles and ultrasound - An effective platform for enhanced macromolecular delivery to representative cells of the retina. PLoS One. 2017;12(5):e0178305.

19. Kwan JJ, Graham S, Myers R, Carlisle R, Stride E, Coussios CC. Ultrasound-induced inertial cavitation from gas-stabilizing nanoparticles. Physical Review E. 2015;92(2):023019.

Thomas RG, Jonnalagadda US, Kwan JJ. Biomedical Applications for Gas-Stabilizing Solid Cavitation Agents. Langmuir. 2019;35(31):10106-15.

### EXAMPLES

### Example 1: Preparation and Characterisation of PLGA Microparticles

### Preparation

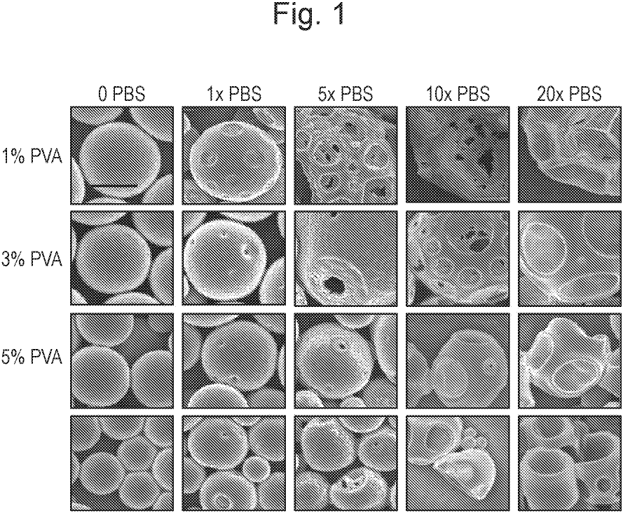

Multi-cavity PLGA microparticles (mcPLGA MPs) were prepared by an adapted water/organic/water double emulsion solvent evaporation process. 50 mg of poly(lactic-coglycolic acid) PLGA was dissolved in 2 mL of dichloromethane (DCM). Then 100 µl of phosphate buffered saline (PBS) was added to the PLGA solution and sonicated (Ultrasonic processor VCX 130, Sonics and Materials Inc., USA) at 100 W for 30 s in an ice bath to form an emulsion. The obtained water-in-oil (W/O) emulsion was poured into a 5% poly(vinyl alcohol) (PVA) solution and homogenized (Ultra Turrax T-25 Ika Labortechnik, Germany) at 12000 rpm over ice for 5 min. Then this particle suspension was stirred at room temperature for 3 h in a chemical fume hood to allow for evaporation of the organic solvent. The PLGA particles were collected by centrifugation at 1,000 G for 5 min, after which they were redispersed and then subjected to three cycles of centrifugation/wash/redispersion. After the final wash, the fresh microparticles were frozen at – 80° C. and then lyophilised in a lyophiliser (Alpha 2-4 LSCbasic, Christ, Germany) for 48 h to achieve a dried powder for long term storage. For drug loaded PLGA, therapeutics was added to the organic solution prior to emulsification. The resulting particles are shown in FIG. 1, and show a broad range of shapes (from smooth to porous) and sizes (from 0.6 µm to 6 µm in diameter), synthesized under different concentrations of PBS and PVA. Alternative formulations investigated using the same method were 0x, 1x, 5x, and 10x PBS, and 1%, 3%, 5%, and 10% PVA. 1x PBS is given as 0.01 M concentration in accordance to the manufacture instructions.

### Rhodamine B (RhB) as a Model Drug for HIFU Enhanced Drug Delivery Study

The model drug, rhodamine B (RhB), was encapsulated in mcPLGA MPs (RhB-mcPLGA MPs) using emulsion solvent evaporation technique mentioned above. The quantity of RhB present was calculated according to the UV-absorbance of RhB at 553 nm measured by a UV-Vis Spectrometer (Shimadzu UV 2450). A standards curve was made in PBS to correlate the mass of RhB in solution with the UV-absorbance spectral curve. The loading efficiency was calculated by first measuring the remaining RhB within in the supernatant of RhB-mcPLGA MPs after solvent evaporation and subtracting it from the total amount of RhB added into the system. This difference was divided by the total amount of RhB added and multiplied by 100 to obtain the percent of RhB loaded.

### Characterisation Technique

Size and surface morphology of the resultant RhB-PLGA particles were assessed using a JEOL JSM-6700 Field Emission Scanning Electron Microscope (FE-SEM; JEOL Ltd., Akishima, Tokyo, Japan) at an acceleration voltage of 5 kV. Samples for the SEM were prepared by dropping 10 µl of 1 mg/ml suspension on silica wafers and air drying. The wafers were mounted onto a metal stub using double-sided electrical tape and coated with platinum (JFC 1600 Auto Fine Coater, JEOL Ltd., Akishima, Tokyo, Japan) for 2 min at 20 mA. All images were recorded under Secondary Electron Imaging (SEI) mode. Size distributions were determined by dynamic light scattering (DLS) (Malvern Nano-ZS, Malvern Panalytical, Malvern, UK).

### Characterisation Results

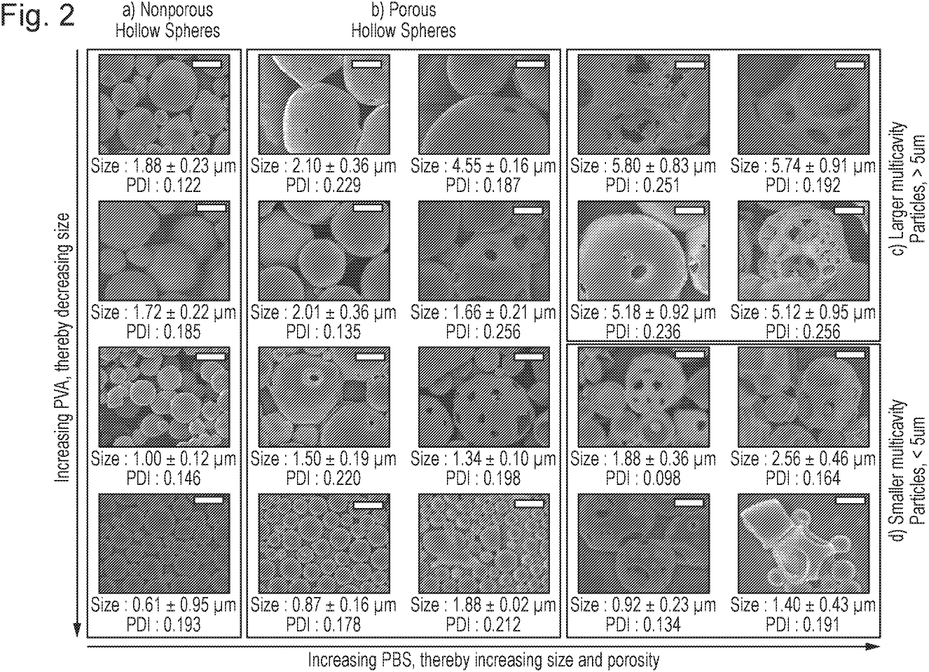

SEM images of PLGA microparticles (FIG. 2) prepared using the double emulsion-diffusion-evaporation method exhibited a multitude of diameters and porosity proportional to the concentration of porosigen (PBS) and stabilizer (PVA) and are been labelled into 4 categories, details of which are provided in the following sections, based on their morphology and diameter namely a) non-porous hollow spheres, b) porous hollow spheres, c) large multi-cavity particles (> 5 µm in diameter), and d) small multi-cavity particles (<5 µm in diameter). For ease of understanding, the type of pore openings were classified either as pores (holes running throughout the thickness) or as cavities (surface indentations not running throughout the thickness of the shell but limited to the surface).

PLGA microparticle formulations without PBS in the internal aqueous phase led to nonporous hollow spheres irrespective of the quantity of stabiliser. Increasing the amount of porosigen in the internal aqueous phase of the water-in-oil-in-water (W/O/W) droplet resulted in hollow spheres with small and infrequent pores. Further increases of PBS concentration led to multi-cavity particles that were not uniformly spherical. Instead, cup shapes, highly porous spheres, and various aspherical shapes were present. In contrast, increasing the amount of stabilizer present in the bulk aqueous phase prior to heating inhibited the presence of pores and decreased the diameter of the polymer particles for all formulations. The population of multi-cavity particles may have porous particles present and vice versa. Thus, these categories are based on the predominant observed structure.

For this study, the multi-cavity particles were separated into two groups based on diameter, i.e., large multi-cavity particles (> 5 µm) and small multi-cavity particles (< 5 µm). This cut-off to distinguish the larger from the smaller variants at 5 µm was chosen so as to distinguish the multi-cavity particles and compare their acoustic response to both setups of HIFU and the diagnostic imaging and then determine their ideal potential use in the different ultrasound regimens. Most commercially available ultrasound contrast agents (UCAs) have a diameter of less than 5 µm in diameter on average, so this enables a comparison to be made with commercially available UCAs, and also enables a study of the different performance of the larger particles. Smaller variants would be ideal for use in the therapeutic domain given their favourable size, and the larger particles would nucleate bubbles with a larger scattering cross section and behave as better contrast agents but achieve poorer perfusion.

### Example 2 - Drug Delivery

### HIFU Setup

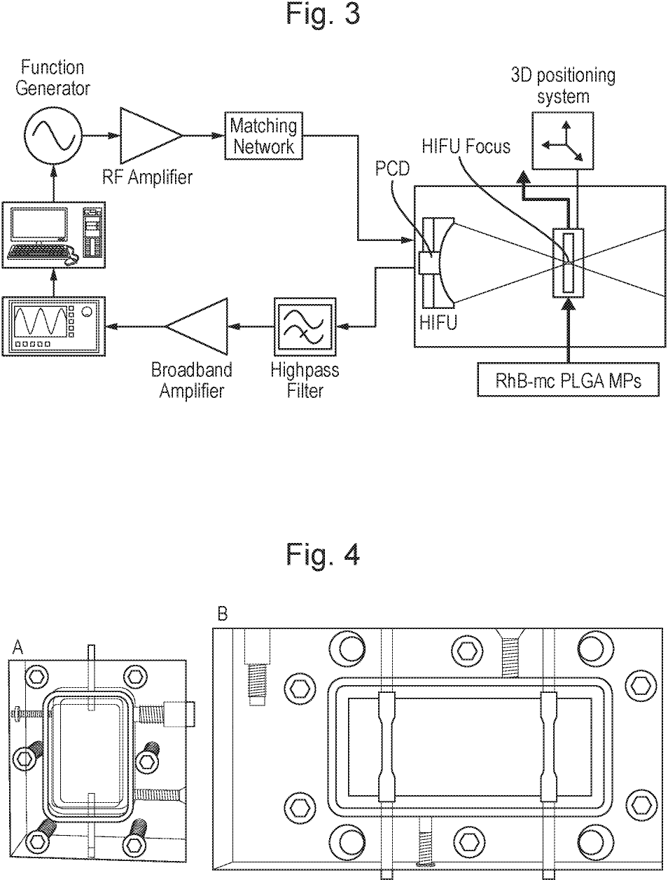

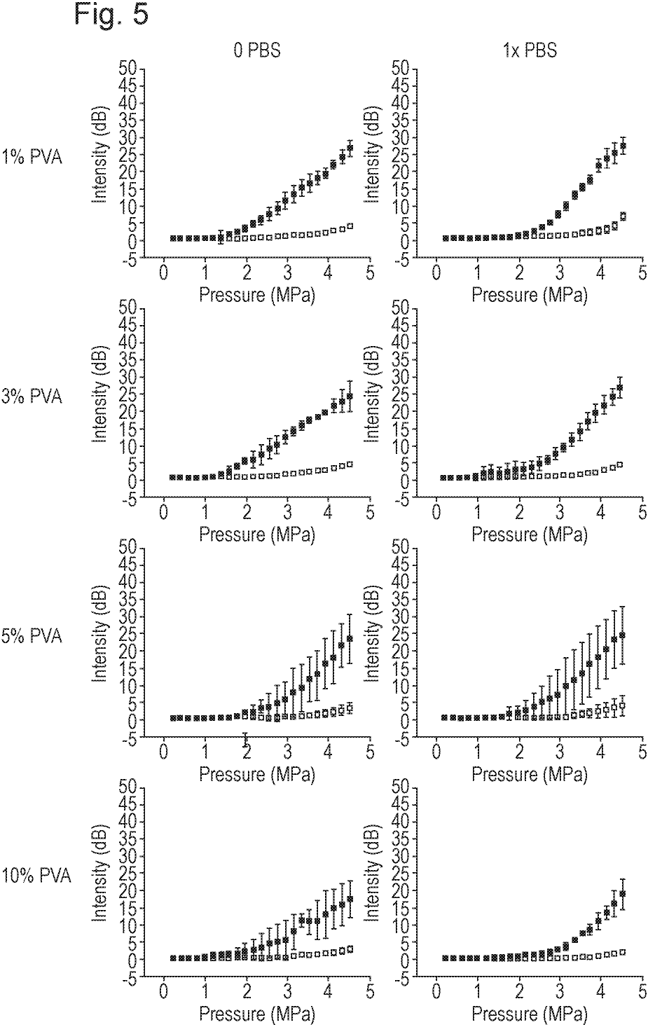

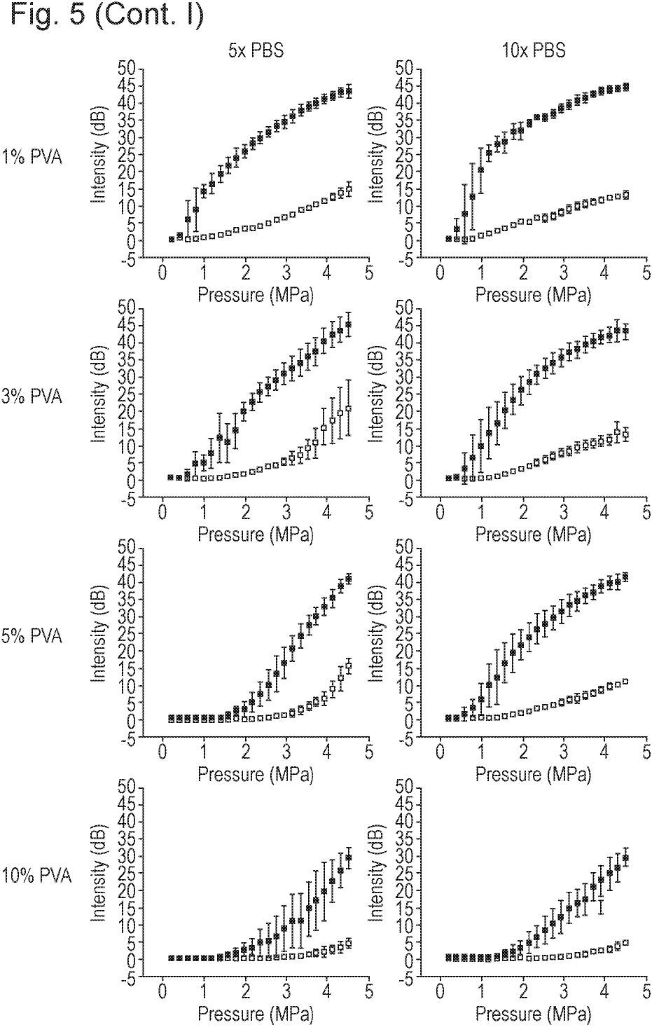

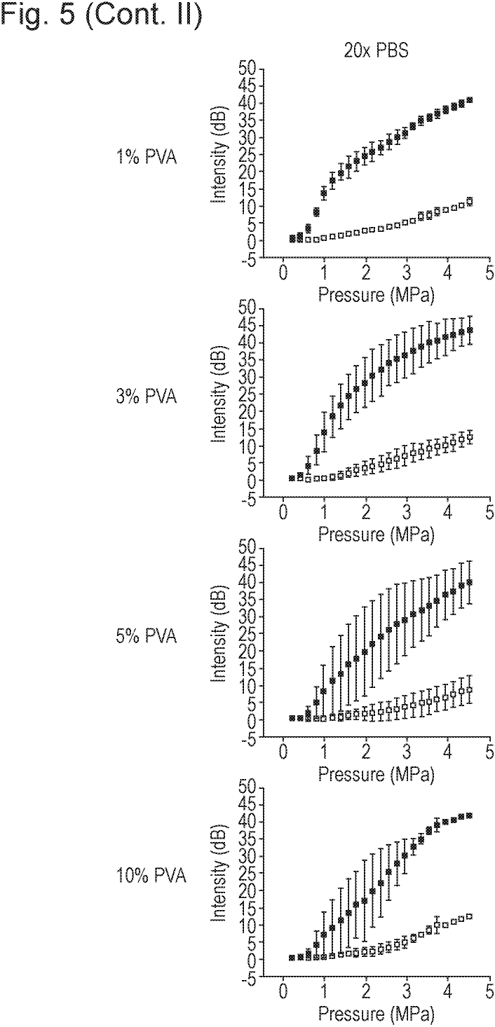

Using a conventional high intensity focused ultrasound (HIFU) setup (FIGS. 3 and 4), the particles of example 1 were exposed to 1.1 MHz ultrasound at various pressure amplitudes. FIG. 5 shows the cavitation intensity (i.e., the likelihood for the suspension of particles to respond to ultrasound) and indicates that the particles respond to HIFU at drastically lower pressure amplitudes as compared to water and spherical variants (following the same production method). These pressure amplitudes are comparable to, if not lower, than those of other polymeric cavitation nucleation agents currently under investigation in other groups.

### HIFU Enhanced Drug Delivery

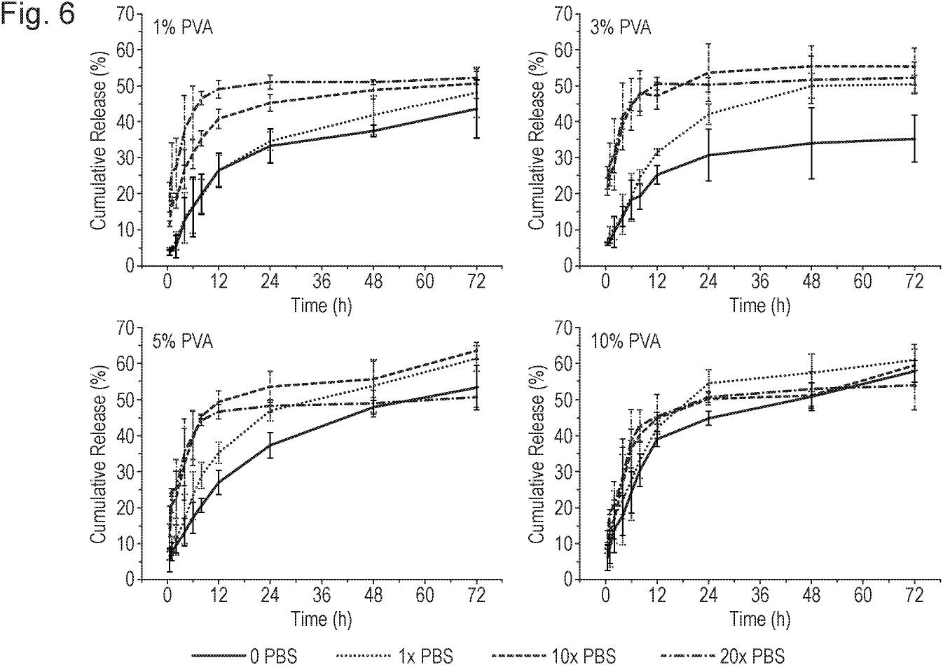

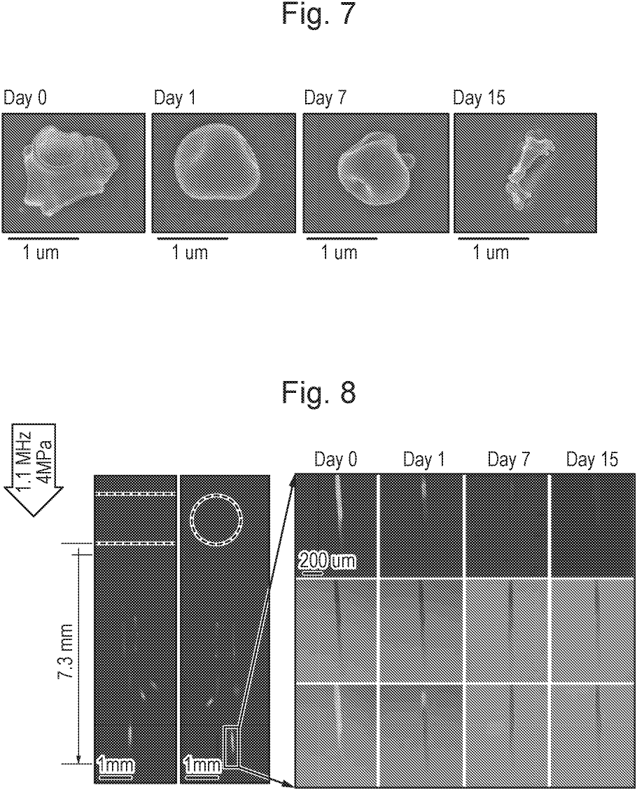

The in vitro release study was performed in PBS buffer solutions using sample and separation method. 50 mg of freeze-dried PLGA/rhodamine was collected and dispersed in 50 ml of 0.01 M PBS (pH 7.4) buffer solution in sealed vials. This solution was maintained at 37° C. under magnetic agitation. At each time point, 1 ml of the solution was taken out and centrifuged at 3000 RCF for 5 min. The concentration of the drug in the collected supernatant was analyzed using UV-visible spectrophotometer at 553 nm. The experiment was performed in triplicate. Results (FIG. 6) show a rapid release of the model drug in 24 hours, likely due to the edges of the cavities dissolving first (FIG. 7). Afterwards, the model drug was slowly released across two weeks.

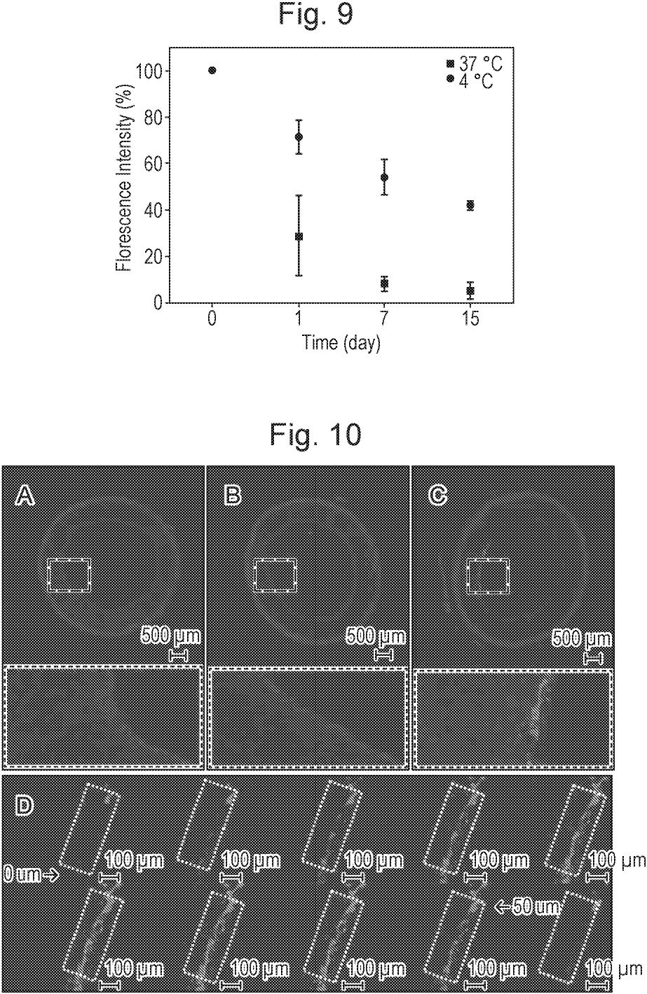

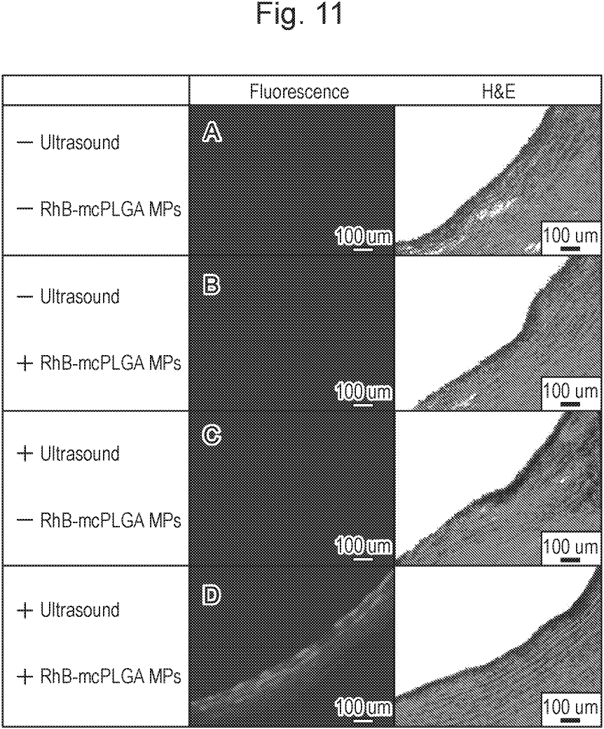

The RhB-mcPLGA MPs were then tested to determine the capability to be embedded into an agarose model. FIG. 8 shows that the particles were implanted into the agarose at depths of up to 7 mm. The particles remained in the agarose for 15 days, slowly releasing the fluorescent dye. This was quantified at both 37 C and 4 C (FIG. 9). Similar experiments were conducted with porcine arteries. FIG. 10 shows that particles were able to penetrate between the intima and media of the artery and remain embedded. Only the region that was targeted shows signs of mcPLGA MPs implantation, suggesting this method is spatially controllable. Furthermore, these particles are not simply on the surface of the slides and are seen throughout the thickness of the targeted area. The process also does not further damage the endothelium (FIG. 11).

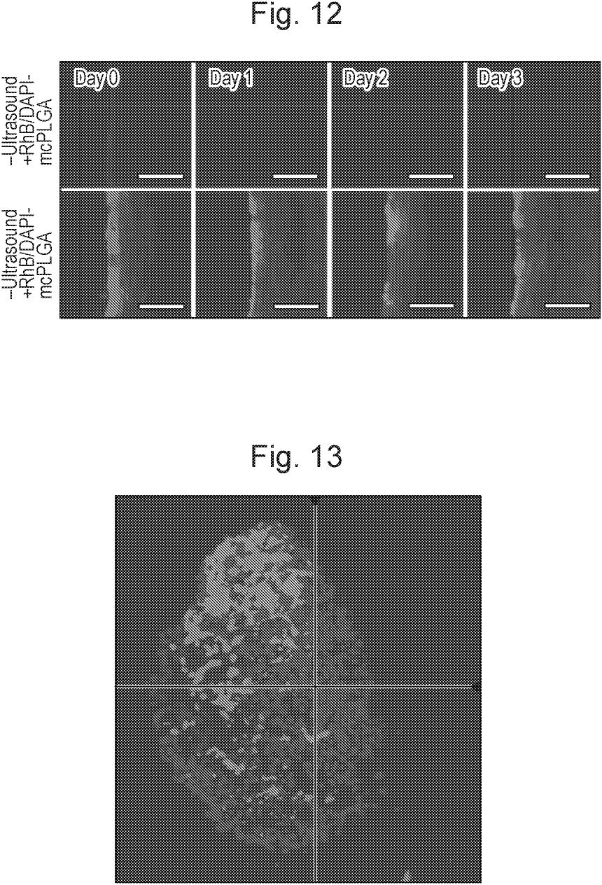

We have also loaded the mcPLGA MPs with both DAPI and RhB, and remotely implanted the loaded particles using HIFU into ex vivo porcine arterial tissue. We monitored the location of both the DAPI and RhB across three days (FIG. 12). Here, RhB labels the location of the particles, whereas DAPI is only fluorescent when bound the DNA. Therefore, DAPI was only observed after release from the particles during degradation, whereby the molecule diffused across the cellular membrane and bound to the DNA of the cell. With each day, DAPI was found to travel further from the initial implantation site.

As the cells in the ex vivo porcine artery were considered non-viable, we have also tested the ability to deliver DAPI to viable cells using a 3D foam cell spheroid model (FIG. 13). Similar to the porcine arteries, the DAPI diffused through the membranes of the cells to stain the DNA of the foam cells. This shows the capability to deliver therapeutics to the cells without damaging the entire structure or integrity of the cell mass.

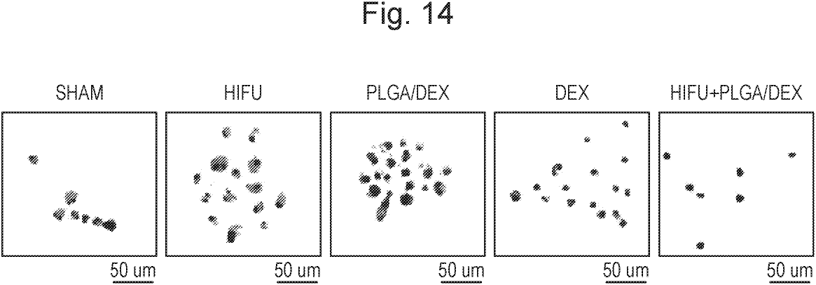

Furthermore, we have also been able to load dexamethasone (Dex) into the mcPLGA MPs and test efficacy of Dex-PLGA MPs in reducing lipid accumulation in foam cell spheroids. To determine the physiological effects of Dex on cholesterol loaded foam cell spheroids, the Oil Red O stain was used to determine the presence of neutral lipids and triglycerides in cells. Lipid was stained in red and was visualised through an optical microscope (FIG. 14). HIFU implanted Dex-PLGA show to be more effective in reducing lipid accumulation.

### Example 3 - Acoustic Characterisation of Microparticles

### Sample Chamber

To make the acoustically transparent agarose sample chamber, 1% (w/v) of agarose solution was boiled and degassed for 30 min. The agarose solution was then poured into a bespoke cuboid mould (50 mm in length × 30 mm in width) and sealed with acoustically transparent mylar windows. A 1.6 mm steel rod was threaded through the mould. The rod was removed after gelation was complete, creating a channel for fluid flow.

### Resuspension of Microparticles

The dry powder, as prepared in Example 1, was resuspended by mixing with deionized water and vortexed briefly (<5 sec) which led to no agglomeration as indicated by a the polydispersity index (PDI) = 0.13 by DLS. The particles were pumped into the acoustic sampling chamber and the containment vessel and no sedimentation was observed for particle diameters < 5 µm. For larger variants, i.e. diameter > 5 µm, sedimentation was observed after 10 min. To prevent this, all experiments were completed within 7 minutes.

### Therapeutic Ultrasound Setup

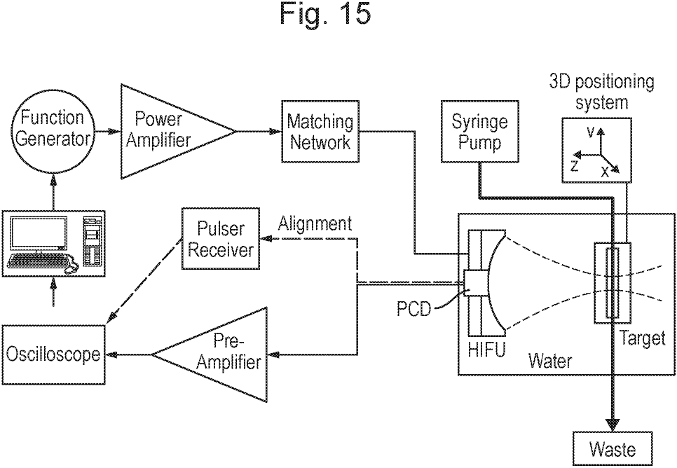

A 1.1 MHz high intensity focused ultrasound (HIFU) transducer (H102, Sonic Concepts, Bothell, WA, USA) was used for acoustic excitation. A 15 MHz passive cavitation detector (PCD) (V319, Olympus, Singapore) — co-axially aligned with the HIFU transducer focus — was used for detection of acoustic emissions at the HIFU focus. The HIFU transducer was calibrated using a 0.2 mm needle hydrophone (SN2562, Precision Acoustics, Dorset, UK). The geometric focus of the transducer was 1.37 mm in width and 10.21 mm in length. The HIFU transducer was driven by a function generator (33210 A, Keysight Technologies, Santa Rosa, CA, USA) and a RF power amplifier (1040 L, Electronics & Innovation, Rochester, NY, USA). All experiments with HIFU were carried out in a large tank filled with filtered, degassed, and deionized water. Acoustic amplitudes in this study were reported in MPa peak negative pressure amplitudes. A schematic representation of the setup is shown in FIG. 15.

### Acoustic Characterization of Microparticles

The agarose phantom sample chamber was submerged in the degassed water tank and aligned to the focus of the transducer. With the channel filled with air, the PCD was driven with a pulser-receiver (JSR Ultrasonics DPR300, Imaginant, Pittsford, NY, USA) to determine the position of the channel. A 3D positioning system was used to adjust the chamber until the channel was at the focus of the HIFU transducer. A 1 mg/ml suspension of microparticles were flowed through the channel using a syringe pump at a rate of 0.2 ml/min for ultrasound exposures. PLGA microparticles were exposed to 20 cycle bursts with increasing peak negative pressure amplitude at a pulse repetition period of 0.1 s. Acoustic emissions from PLGA microparticles were detected using a 15 MHz PCD co-axially aligned with the HIFU transducer. The PCD output was amplified using a broadband preamplifier (SR445A, Stanford Research Systems, Sunnyvale, CA, USA). The received signals were then recorded onto an oscilloscope (DXOX3032A, Keysight Technologies, Santa Rosa, CA, USA) and post processed to determine the power spectral density (PSD) curve. For each burst, the area under the PSD curve was determined and compared to degassed water exposed to HIFU under the same conditions. Following the signal processing using MATLAB R2019b cavitation was considered to have occurred if the received signals were 6 dB higher than noise from the water control. The probability of cavitation was determined as the percentage of bursts that recorded a cavitation event out of the total number of HIFU bursts (120 bursts).

### Cavitation Threshold Determination

To estimate the cavitation threshold, a sigmoid function was fit to the probability for both harmonic and broadband signal. The sigmoid fitting function is defined in eq. 1:

$$f = \frac{1}{1 + {\text{e}}^{\text{-} \left(\left(p \text{-} {p}_{50}\right) k\right)}} \qquad \text{(1)}$$

Where, f is the probability for cavitation, p is the input pressure, p₅₀ is the cavitation threshold defined as the pressure amplitude value for achieving in 50% of the total number of pulses contained a cavitation, k is the slope of the fit. This function was fit to the experimental data by minimising the sum of square residuals using Microsoft Excel.

### Cavitation Response

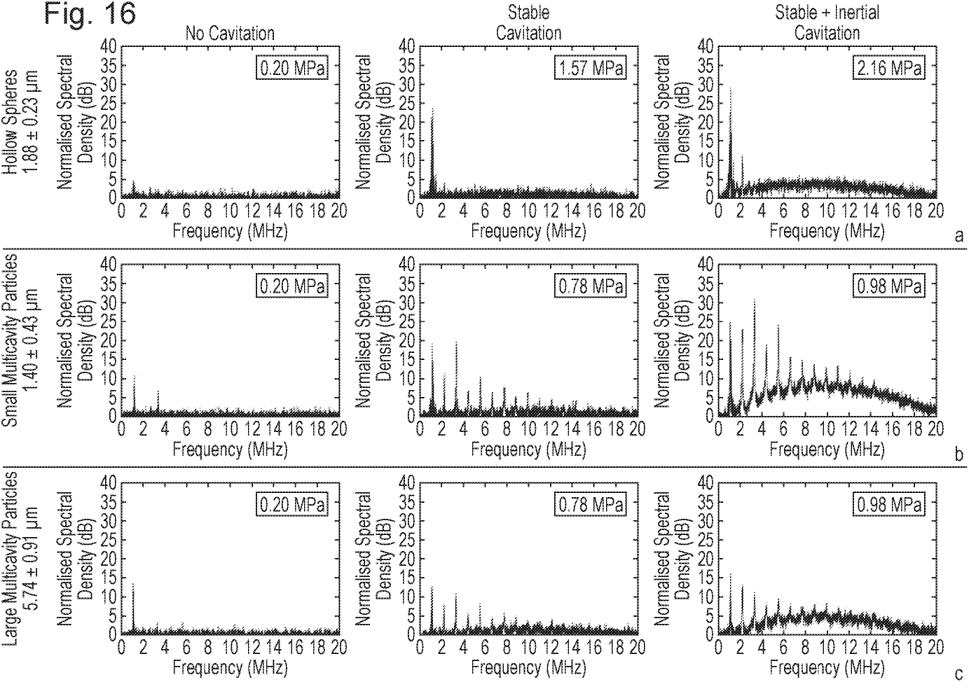

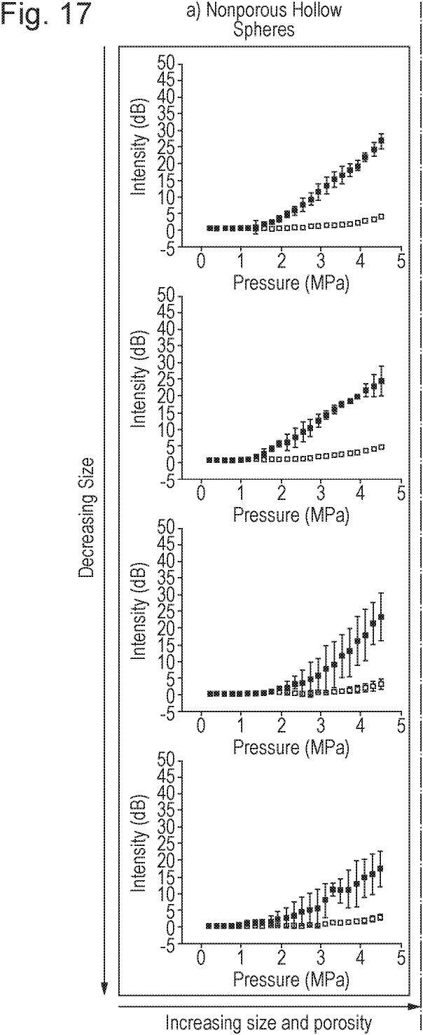

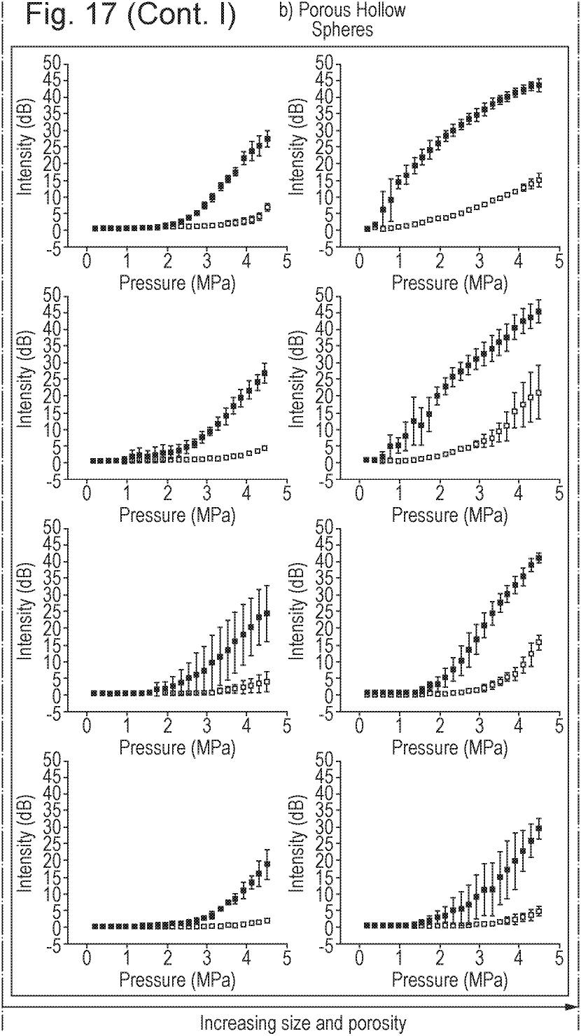

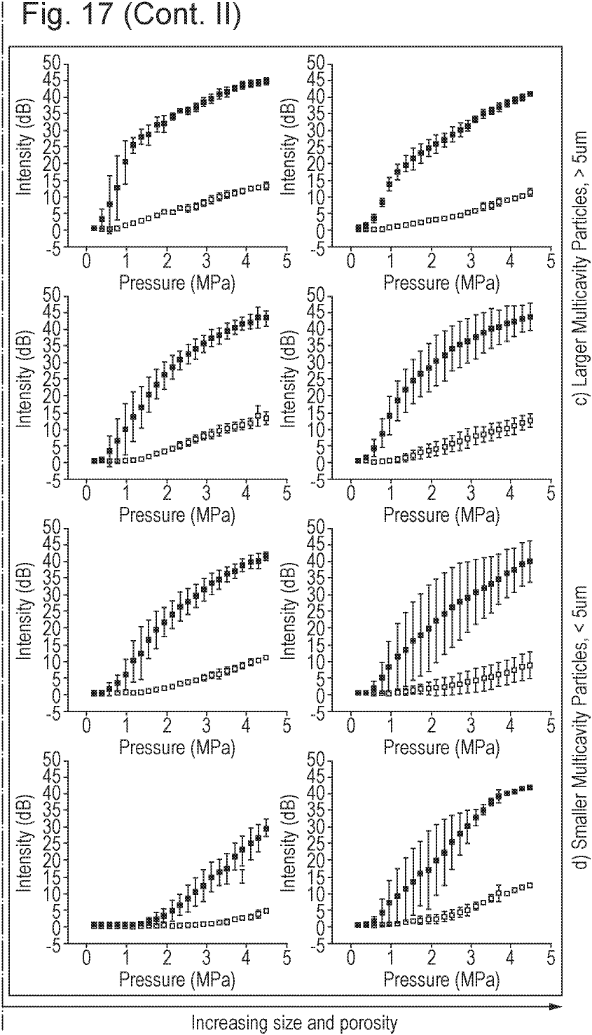

FIG. 16 shows representative images of normalized PSD curves for three different shapes of particles, namely the hollow spheres, small multi-cavity, and large multi-cavity microparticles. Cavitation was detected for all types of microparticles. Although the presence of harmonic emissions was observed for all microparticle formulations, substantial broadband emissions were only present for some of the formulations and was dependent on both the diameter of the microparticle and acoustic intensity (FIG. 17). Broadband emissions, if present, only became apparent at pressure amplitudes larger than the pressure amplitudes required for harmonic emissions.

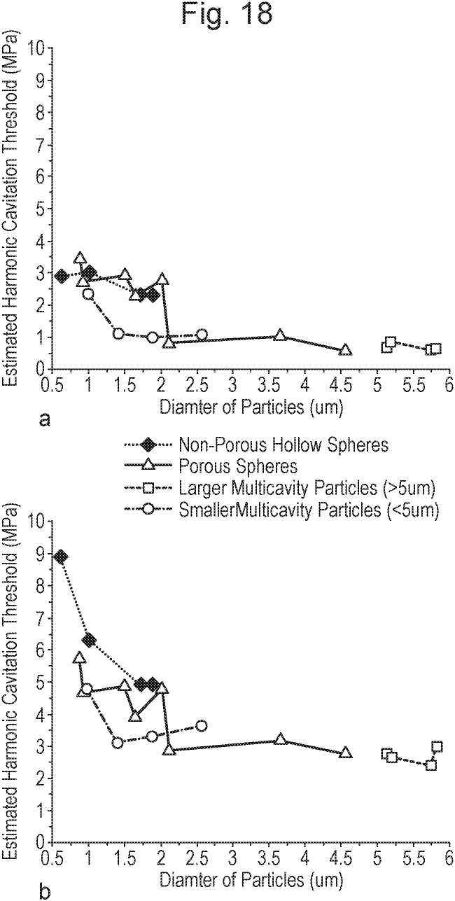

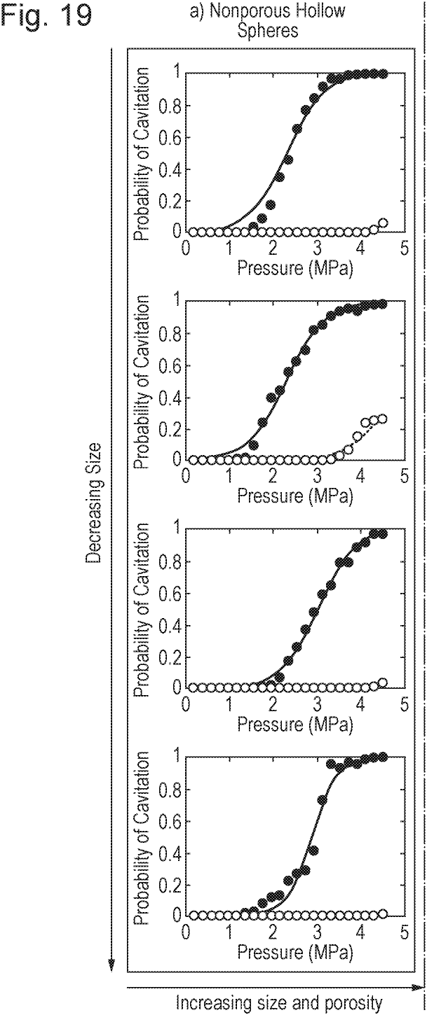

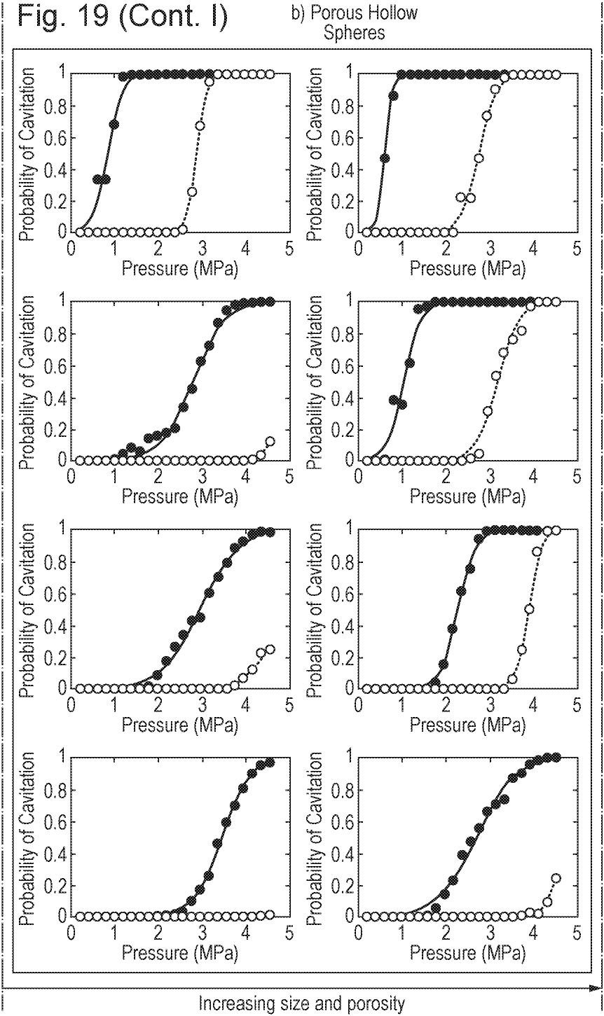

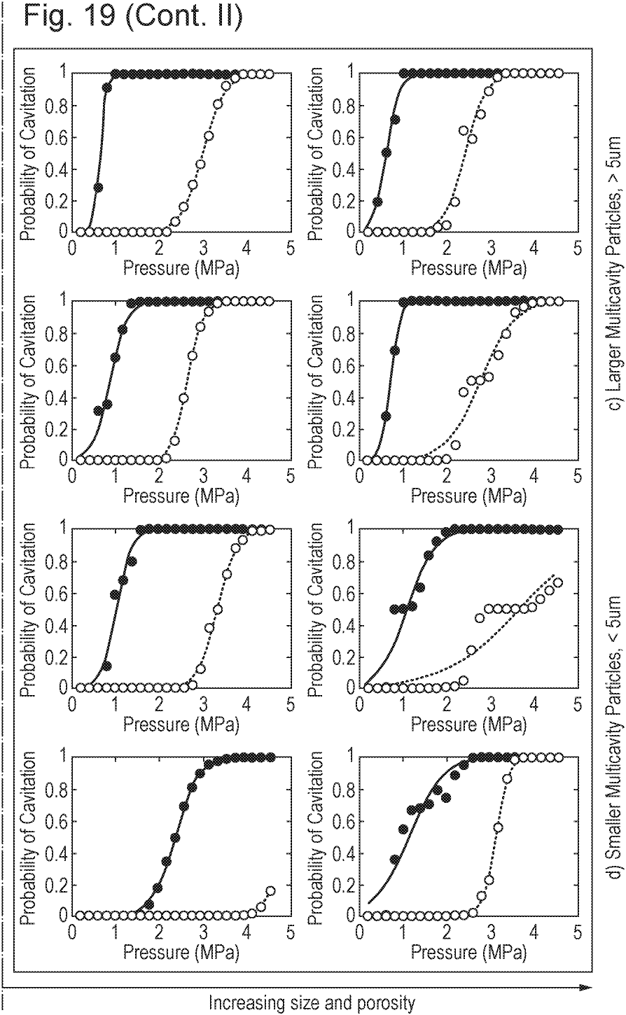

FIG. 18 shows the estimated harmonic and broadband cavitation thresholds determined by the probability of cavitation (FIG. 19) for all the microparticles tested. Both harmonic and broadband thresholds were governed by the diameter and shape of the microparticles. Irrespective of shape, larger microparticles had lower cavitation thresholds. This trend was most evident for the onset of broadband noise. Regarding the shape of the microparticles, there was generally a lower cavitation threshold for both harmonic and broadband emissions for porous particles compared to smooth hollow spheres. Similarly, more porous particles, i.e., multi-cavity microparticles as opposed to surface pores on spheres, emitted harmonic and broadband noise at lower input pressures; larger cavities nucleated cavitation at the lowest acoustic intensity.

Our results for the inertial cavitation threshold for the hollow spheres indicated that a pressure range between 4.5 MPa to 9 MPa was required to achieve 50% probability of inertial cavitation. As the size decreased for each morphology group, a higher input pressure was required to achieve 50% probability of harmonic cavitation.

### Example 4: Contrast Enhancement

### Diagnostic Acoustic Set-Up

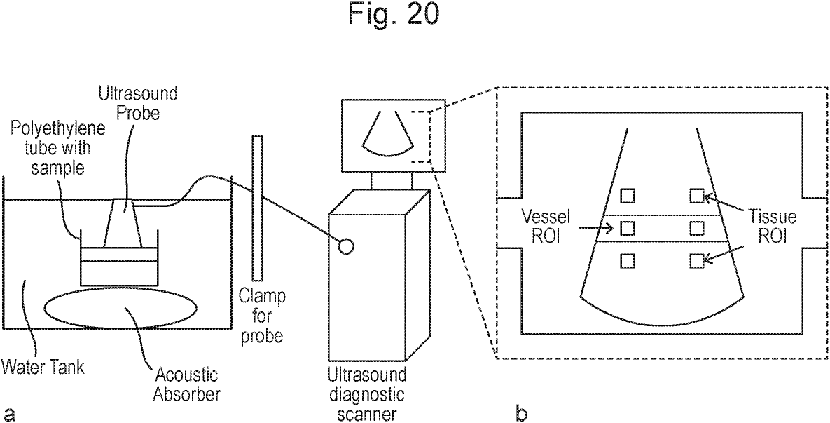

An E-Cube 12-R (Alpinion Medical Systems, Seoul, South Korea) clinical ultrasound imaging system with a linear array transducer (L3-12, Alpinion Medical Systems, Seoul, South Korea) was used to acquire images at the focal zone depth (5 cm) at a 12 Hz framerate. Scanning was performed with B mode operating at 10 MHz. Additionally, the mechanical index of this scanner was 1.1 at a 100% acoustic power giving a peak negative pressure of 3.2 MPa 99. Data was saved in triplicate for each sample. Three independent samples for each formulation were tested. A schematic representation of the diagnostic ultrasound setup is shown in FIG. 20a.

### Contrast Enhancement Measurements

For contrast enhancement measurements, a flow system was implemented using an acrylic water bath, a syringe pump (KD Scientific, Holliston, MA, USA), and flexible lowdensity polyethylene tubes (outer diameter 2.42 mm, thickness 0.37 mm). A dose of 6 mL reconstituted PLGA particles (1 mg/ml) were infused into the phantom holder via a syringe pump at a constant rate of 1 ml/min. The sample holder was placed in a water bath and the probe was placed directly above the vessel. As a control, deionized water was also run through the sample chamber and saved. The data was saved in triplicate in B-Mode. Afterwards contrast to tissue ratio (CTR) analysis was performed using ImageJ 1.52q (National Institutes of Health, Bethesda, MD, USA) to quantify the ability of each PLGA particle sample to distinguish between vessel and tissue using eq 2:

$$C T R = \frac{2 {\left({μ}_{t} \text{-} {μ}_{v}\right)}^{2}}{{\left({σ}_{t} {}^{2} \text{-} {σ}_{v} {}^{2}\right)}^{2}} \qquad \text{(2)}$$

where µₜ and µᵥ represent the mean backscatter signal strength in the tissue and within the vessel lumen region, respectively, while σₜ², and σᵥ² represent the corresponding variances. Four region-of-interests (ROIs) within the tissue and two ROIs within the vessel were selected. Each ROI was a 0.5×0.5 mm square. Images were acquired in triplicate for each sample using the linear array probe. The mean signal was averaged across all tissue and vessel ROIs to reduce variability. The four tissue ROIs were selected along the same horizontal and vertical axes as the vessel ROIs (as shown in FIG. 20b).

### Contrast Enhancement Results

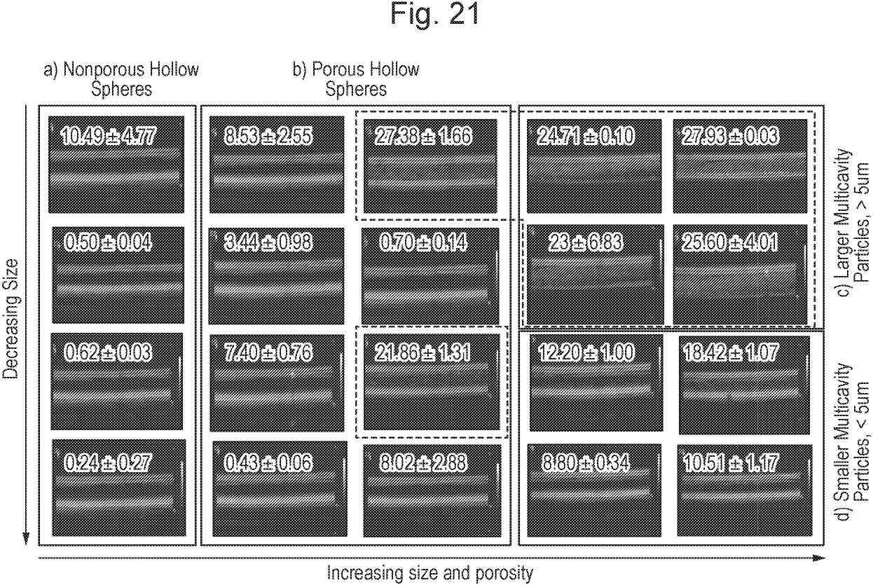

The representative images for all the microparticle formulation samples tested with the diagnostic ultrasound setup (as described above) at maximum input power, along with their calculated CTR values are shown in FIG. 21. Generally, smaller and smoother microparticles had lower CTR values compared to more porous particles. The highest CTR values corresponded with larger multi-cavity particles (5.12 µm to 5.18 µm in diameter).

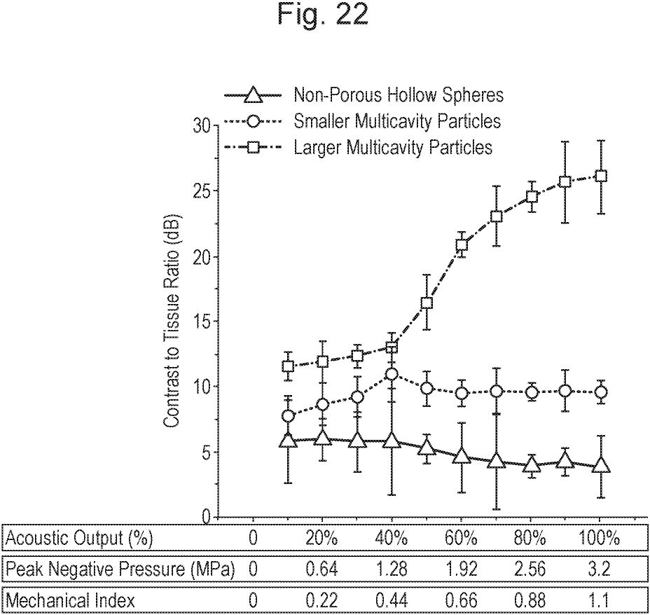

FIG. 22 shows the measured CTR for representative microparticles from the different morphology groups (2 µm in diameter smooth spheres, 2 µm in diameter multicavity microparticles, and 6 µm in diameter multi-cavity microparticles) in addition to deionized water for increasing input pressures from 10% to 100% power (corresponding MI values of 0.11 to 1.1). The CTR of the 2 µm in diameter smooth spheres remained at 6 dB for all input powers tested. Smaller multi-cavity microparticles provided CTR values greater than the smooth spheres at all input powers and displayed a subtle increase in CTR for input powers greater that 40%. Larger multi-cavity microparticles consistently delivered the highest CTR values for all powers tested. Similar to the smaller multi-cavity particles but to a greater extent, the CTR of the larger multi-cavity particles increased with increasing input power.

For all PLGA microparticle formulations tested to emit detectable harmonic noise in response to therapeutic ultrasound, was unexpected. Such results are indicative of stable cavitation.

### Example 5 - Contrast Enhancement of Drug Delivery Particles

### Preparation

As in Example 4, an E-Cube 12-R (Alpinion) with a L3-12 transducer was used to acquire images at the focal zone depth (5 cm) at a 12 Hz framerate. Scanning was performed with B mode operating at 8.5 MHz. Additionally, the mechanical index of this scanner was 1.1 giving a peak negative pressure of .47 MPa.

An acoustically transparent agarose sample chamber was made from a 3% (w/v) of agarose solution, which was boiled and degassed for 30 min to prevent cavitation as a result of endogenous bubbles. The agarose solution was then poured into a bespoke cuboid mold (50 mm in length × 30 mm in width) and sealed with acoustically transparent mylar windows. A 1.6 mm steel rod was threaded through the mold. After gelation was completed, the rod was removed, creating a flow channel.

### Data Collection

For this, a flow system was implemented using an acrylic water bath, a KD Scientific syringe pump (MA, USA), and flexible PVC tubes. A dose of 6 mL reconstituted PLGA particles were infused into the phantom holder via a syringe pump at a constant rate of 1ml/min. The sample holder was placed in a water bath and the probe was placed directly above the vessel. All samples were tested at a concentration of 1 mg/ml in this setup. The acoustic power was set at 60% as prior results showed highest enhancement at 60% power for test samples. Images were saved in triplicate for each sample. As a control, deionised water was also run through the sample chamber and saved.

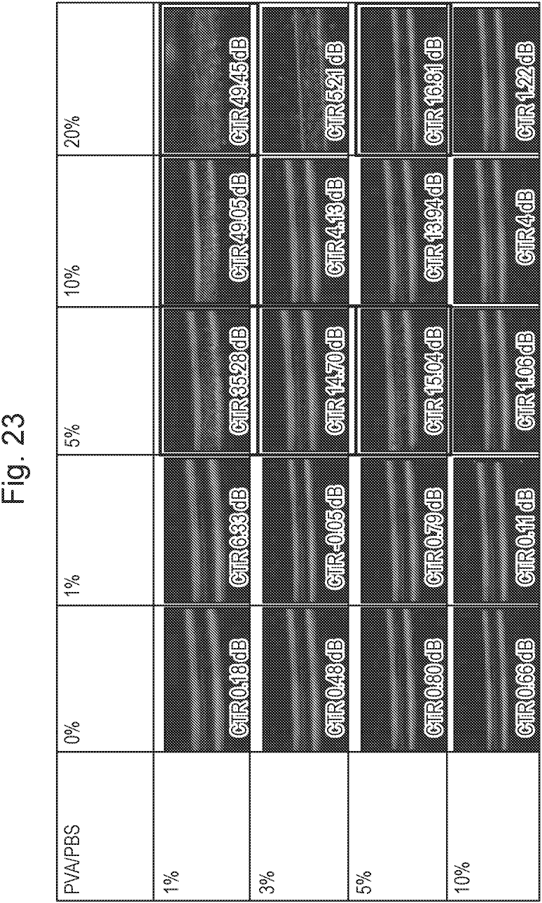

As in Example 4, the data was saved in triplicate in B-Mode and save as beamformed data. Afterwards contrast to tissue ratio (CTR) analysis was performed to quantify the ability of each PLGA particle sample to distinguish between vessel and tissue using the following equation (eq. 2):

$$\text{CTR =} \frac{2 {\left(μ \text{} \text{t} - μ \text{} \text{v}\right)}^{2}}{{\left(σ \text{t*2} - σ \text{v*2}\right)}^{2}}$$

where µₜ and µᵥ represent the mean backscatter signal strength in the tissue and within the vessel lumen region, respectively, while σₜ², and σᵥ² represent the corresponding variances. Four region-of-interests (ROIs) within the tissue and two ROIs within the vessel were selected. Each ROI was a 1×1 mm square. Images were acquired in triplicate for each waveform at the optimized acoustic output using the linear probe. The mean signal was averaged across all tissue and vessel ROIs to reduce variability. The four tissue ROIs were selected along the same horizontal and vertical axes as the vessel ROIs (as shown in FIG. 23)

[image]

### Results

The results for the CTR analysis and representative images are shown in FIG. 23. As can be seen and as expected, highest CTR values were obtained for lesser PVA and higher PBS concentrations. Increasing PVA decreases the size and increasing PBS increases the porosity of these particles, thereby increasing the number of cavities on the surface. CTR values as high as 49 dB were achieved, although with relatively larger particles (diameter > 5um; 1% PVA 10x PBS). However, the clinically relevant particle sizes (diameter<2 um) were 5% PVA 10x PBS having a CTR of 13.94 dB. These particles achieved enhancement comparable to the commercially available ultrasound contrast agents (CTR values 10-20 dB). These results indicate that these particles are capable of providing contrast enhancement required for clinical imaging and can be used as both drug delivery agents and contrast agents.

### Example 6: Subharmonic Imaging of Microparticles

The large multi-cavity particles (> 5 µm) and small multi-cavity particles (< 5 µm), as defined in Example 1, were further imaged using sub-harmonic imaging at various MI.

A modified Logiq 9 ultrasound scanner with a 4C curvilinear probe was used to acquire subharmonic data transmitted at 2.5 MHz, and received subharmonic signals at 1.25 MHz.

Contrast signals were measured using a 2.25 L water tank. The water tank was also equipped with an acoustic window made out of this plastic (thickness: 1.5 mm). An inlet on the top of the tank was constructed for injecting microparticles.

The dry powder, of example 1, was re-suspended by mixing with deionised water and vortexed briefly (<5 seconds). The scanner was then used to capture cine clips in triplicate following the injection of 2 ml of the microparticles (concentration of 1 mg/ml) per litre of saline. The mixture was kept homogenous by the use of a magnetic stirrer.

Large multi-cavity particles (in this case 5 µm), small multi-cavity particles (in this case 2 µm) and hollow spheres were all imaged in regular B mode and with the Logiq 9, at an MI of 1.4 and 0.4.

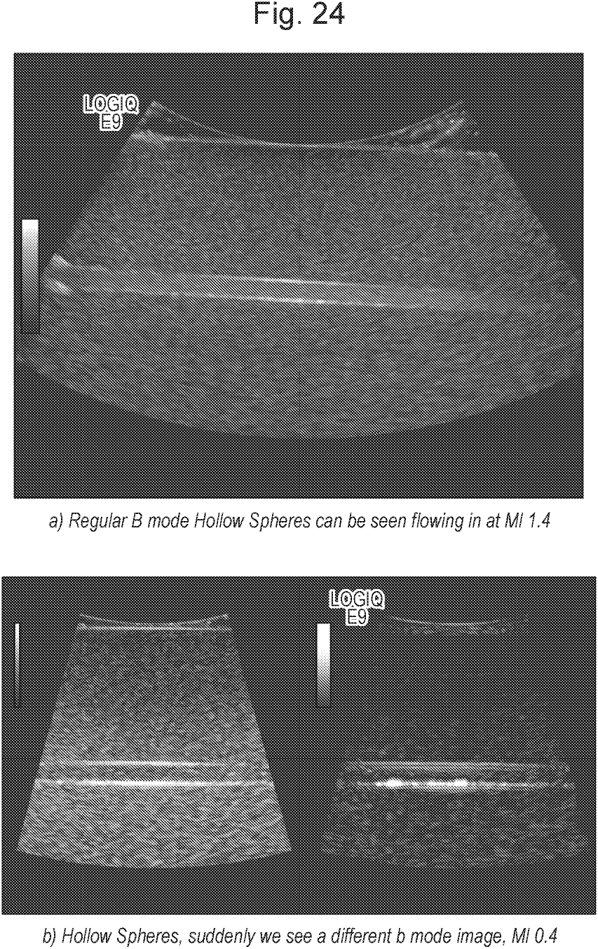

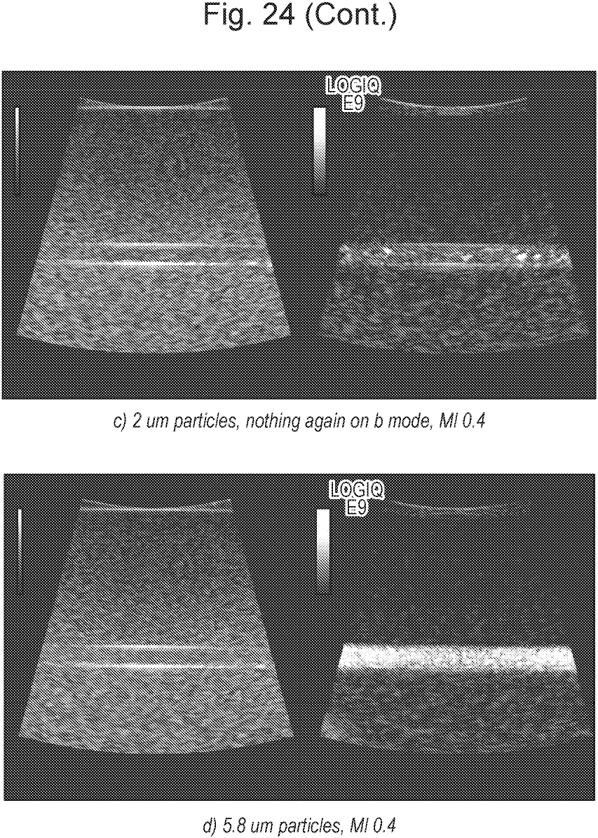

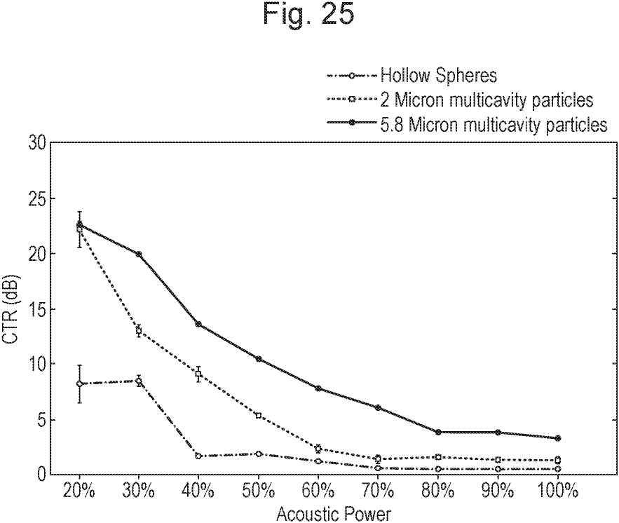

Results showed that even at an extremely low MI of 0.4, particles could be imaged using this sub-harmonic imaging technique. An especially clear response was observed for the large multi-cavity particles, as can be seen in FIG. 24. A comparison of the CTR of different sized particles at different acoustic power levels is shown in FIG. 25.

Imaging at this MI is desirable as lower MIs indicate a lower chance of adverse effects on the body due to the ultrasound.

1. A core-shell micro-particle comprising a biodegradable polymer with at least two or more surface cavities.

2. The core-shell micro-particle according to claim 1, wherein the biodegradable polymer is an aliphatic polyester (including but not limited to poly(lactic-co-glycolic acid) (PLGA), polylactic acid (PLA), polyglycolic acid (PGA), polycaprolactone (PCL), poly(butylene succinate) and its copolymers, poly(p-dioxanone) (PPDO), poly(hydroxybutyrate) (PHB), and polycarbonates), aromatic copolyester (including but not limited to poly(butylene adipate-co-terephtalate) (PBAT)), polyamide and poly(ester-amide), polyurethanes, polyanhydrides, polysaccharides, (including but not limited to chitosan, cellulose, and hyaluronic acid), and blends or copolymers of the aforementioned examples.

3. The core-shell micro-particle according to claim 1 or 2, wherein the surface cavities are indentations on the shell, and/or form hierarchical porous shell with the hollow core.

4. The core-shell micro-particle according to any one of claims 1 to 3, wherein the shell may comprise of hydrophobic chemicals such as drugs like sirolimus, steroids, dexamethasone, etc.

5. The core-shell micro-particle according to any one of claims 1 to 4, wherein the core comprises a hydrophilic drugs like peptides, proteins, and vaccines provided that the surface cavities do not form tunnels to the core.

6. The core-shell microparticle according to any one of claims 1 to 5, wherein the microparticle has an average diameter of 5 to 10 µm.

7. The core-shell microparticle according to claim 6, wherein the microparticle has an average diameter of 5 to 6 µm.

8. An ultrasound contrast agent comprising a microparticle according to any one of claims 1 to 7.

9. Use of the core-shell micro-particle according to any one of the claims 1 to 7, or the ultrasound contrast agent of claim 8, in drug delivery, contrast enhancement, subharmonic imaging enhancement, theranostics, and/or any combination of the aforementioned applications.

10. The use according to claims 9, wherein the core-shell micro-particle is
(a) introduced into a blood vessel (e.g., through intravenous injection, local deliver, intramuscular injection, catheter injection, etc.) or topically (e.g., through a cream, gel, etc.); and
(b) subjected to a pressure wave (e.g., ultrasound, focused ultrasound, shockwaves, etc.) such that the core-shell micro-particle is embedded into biological tissue (e.g., the wall of the blood vessel, skin, tumor, etc.).

11. The use of claim 9 or claim 10, wherein the core-shell microparticle or the ultrasound contrast agent is used as an ultrasound contrast agent.

12. The use of claim 9 or claim 10, wherein the core-shell microparticle or the ultrasound contrast agent is used as a subharmonic imaging contrast agent.

13. A method of drug delivery comprising
(c) administering an effective amount of the core-shell micro-particle according to any one of the claims 1 to 7, which core shell microparticle contains one or more drugs; and
(d) subjecting the core-shell microparticle or ultrasound contrast agent to a pressure wave at the site for drug delivery, such that the core-shell microparticle or ultrasound contrast agent is embedded into biological tissue at the site.

14. A method according to claim 13, wherein the administration is to a blood vessel or to the skin.

15. A method according to claim 13 or 14, wherein the method further comprises subjecting the site to ultrasound imaging.

16. A method of ultrasound contrast enhancement comprising
(a) delivering the core-shell micro-particle according to any one of claims 1 to 7, or the ultrasound contrast agent of claim 8, to a site which is to be imaged; and
(b) subjecting the site to ultrasound imaging.

17. The method of claim 16, wherein the delivery step comprises introducing the core-shell microparticle or ultrasound contrast agent into a blood vessel or onto the skin, and subjecting the core-shell microparticle or ultrasound contrast agent to a pressure wave such that the core-shell microparticle or ultrasound contrast agent is embedded into biological tissue.

18. The method of any one of claims 15 to 17, wherein the imaging technique is subharmonic imaging.

19. The method of any one of claims 15 to 18, wherein the subharmonic imaging is carried out with an MI of 1.4 or less.

20. The method of any one of claims 15 to 19 wherein the subharmonic imaging is carried out with an MI of 0.8 or less.

21. The method of any one of claims 16 to 20, wherein the core-shell microparticle or ultrasound contrast agent comprises one or more drugs, and wherein the one or more drugs are delivered to the biological tissue in which the core-shell microparticle or ultrasound contrast agent is embedded.
